(19) United States
(12) Patent Application Publication
(10) Pub. No.: US 2023/0372663 A1
(43) Pub. Date: Nov. 23, 2023

(54) SYSTEM AND METHOD FOR ANALYZING SLEEPING BEHAVIOR

(71) Applicant: Dream Team Baby, Corp, Washington, DC (US)

(72) Inventors: Conner Wyatt HERMAN, Washington, DC (US); Abilash MENON, Boxborough, MA (US)

(73) Assignee: Dream Team Baby, Corp, Washington, DC (US)

(21) Appl. No.: 17/750,142

(22) Filed: May 20, 2022

(51) Int. Cl. A61M 21/02

(52) U.S. Cl. CPC A61M 21/02; A61M 2205/3344; A61M 2205/3368; A61M 2205/3303; A61M 2205/3306; A61M 2205/3375; A61M 2230/63; A61M 2205/50; A61M 2205/502; A61M 2205/18

(57) ABSTRACT

A sleeping application receives initial sensor data from one or more sensors of a sensor set in a physical environment for a time period, wherein the sensor set includes at least one of a temperature sensor, a pressure sensor, a humidity sensor, a light sensor, a sound sensor, a thermal-imaging sensor, and a motion sensor. The sleeping application behavior patterns of a set of sleep events of a target subject based on the initial sensor data. The sleeping application generates a recommendation based on the behavior patterns to achieve a target outcome for a target subject in the physical environment. The sleeping application provides the recommendation.

### BACKGROUND

Commercial versions of baby monitors capture images and sounds of babies and provide video and/or audio feeds to parents.

Some sensors are incorporated into wearables and attempt to capture more data about the baby including their movements, heartbeat, oxygen levels, etc. These wearables have limitations and challenges as they need to be either attached to the baby or to the baby's clothing. The wearables may be irritating, easily dislodged, and often collect data that is irrelevant to the behavior of the baby.

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

A computer-implemented method comprises: receiving initial sensor data from one or more sensors of a sensor set in a physical environment for a time period, wherein the sensor set includes at least one of a temperature sensor, a pressure sensor, a humidity sensor, a light sensor, a sound sensor, a thermal-imaging sensor, a camera, and a motion sensor. The method further includes determining behavior patterns of a set of sleep events of a target subject based on the initial sensor data. The method further includes generating a recommendation based on the behavior patterns to achieve a target outcome for the target subject in the physical environment. The method further includes providing the recommendation.

In some embodiments, the method further includes determining if the recommendation was followed and responsive to determining that the recommendation was followed, determining whether the target behavior was achieved. In some embodiments, the method further includes responsive to determining that the target behavior was achieved, updating the behavior patterns based on subsequent sensor data and updating the target outcome based updating the behavior patterns. In some embodiments, the method further includes determining if the recommendation was followed and responsive to the determining that the recommendation was not followed, providing an offer of a reward if the recommendation is subsequently followed. In some embodiments, the sensor set includes the thermal-imaging sensor and the method further comprises: detecting, based on the initial sensor data, the target subject, one or more people near the target subject, and one or more objects in the physical environment; determining, based on the initial sensor data, a distance between the target subject and the one or more people; determining a movement pattern of the target subject in the physical environment; determining one or more movement patterns corresponding to the one or more people; and determining one or more movement patterns for the one or more objects; where determining the behavior patterns is based on the movement pattern of the target subject, the one or more movement patterns corresponding to the one or more people, and the one or more movement patterns for the one or more objects. In some embodiments, the sensor set includes the sound sensor and the method further comprises: detecting, based on the initial sensor data, a sound level in the physical environment; detecting, based on the initial sensor data, sounds of the target subject, one or more people near the target subject, and other sounds in the physical environment; filtering out specific sounds; and determining sound patterns; where determining the behavior patterns is based on the sound patterns. In some embodiments, the sensor set includes the motion sensor and the method further comprises: detecting, based on the initial sensor data, motion of the target subject, one or more people near the target subject, and one or more objects in the physical environment; and determining a target subject movement pattern, a people movement pattern, and one or more object movement patterns; where determining the behavior patterns is based on the target subject movement pattern, the people movement pattern, and the one or more object movement patterns. In some embodiments, the method further includes providing a user interface that requests user preferences about the target outcome, wherein the target outcome is defined based on the user preferences. In some embodiments, the method further includes: receiving subsequent sensor data for a subsequent time period; determining, based on a comparison of the subsequent sensor data to the behavior patterns, that an action is likely to precipitate a nighttime arousal or a naptime arousal; and providing a warning that the action is likely to precipitate the nighttime arousal or the naptime arousal. In some embodiments, the recommendation is provided to a user that is different from the target subject and the method further comprises: determining behavior patterns of a set of sleep events of the user based on the initial sensor data. In some embodiments, the method further comprises receiving the initial sensor data associated with the user that identifies a length of time when the user is asleep and providing a user interface to the user that includes the length of time when the user is asleep as compared to when the target subject is asleep. In some embodiments, the method further comprises providing the initial sensor data as input to a trained machine-learning model and outputting, using the trained machine-learning model, the recommendation for achieving the target outcome.

Embodiments may further include a computing device comprising one or more processors and a memory coupled to the one or more processors, with instructions stored thereon that, when executed by the processor, cause the processor to perform operations comprising: receiving initial sensor data from one or more sensors of a sensor set in a physical environment for a time period, wherein the sensor set includes at least one of a temperature sensor, a pressure sensor, a humidity sensor, a light sensor, a sound sensor, a thermal-imaging sensor, and a motion sensor; determining a baseline for one or more of a set of sleep events of a target subject based on the initial sensor data.

In some embodiments, the operations further comprise: determining behavior patterns of a set of sleep events of a target subject based on the baseline for the one or more of the set of sleep events and the initial sensor data; generating a recommendation based on the behavior patterns to achieve a target outcome for a target subject in the physical environment, providing the recommendation, determining if the recommendation was followed and responsive to determining that the recommendation was followed, determining whether the target behavior was achieved. In some embodiments, the operations further comprise: responsive to determining that the target behavior was achieved, updating the behavior patterns based on subsequent sensor data updating the target outcome based updating the behavior patterns. In some embodiments, the operations further comprise: determining if the recommendation was followed and responsive to the determining that the recommendation was not followed, providing an offer of a reward if the recommendation is subsequently followed.

Embodiments may further include a non-transitory computer readable medium that includes instructions stored thereon that, when executed by one or more computers, cause the one or more computers to perform operations comprising: receiving initial sensor data from one or more sensors of a sensor set in a physical environment for a time period, wherein the sensor set includes at least one of a temperature sensor, a pressure sensor, a humidity sensor, a light sensor, a sound sensor, a thermal-imaging sensor, and a motion sensor; determining behavior patterns of a set of sleep events of a target subject based on the initial sensor data; generating a recommendation based on the behavior patterns to achieve a target outcome for the target subject in the physical environment; and providing the recommendation.

In some embodiments, the operations further comprise: determining if the recommendation was followed and responsive to determining that the recommendation was followed, determining whether the target behavior was achieved. In some embodiments, the operations further comprise: responsive to determining that the target behavior was achieved, updating the behavior patterns based on subsequent sensor data updating the target outcome based updating the behavior patterns.

The specification advantageously describes a non-image-based system that monitors the behavior of a target subject over a sustained period of time based on initial sensor data. The system determines behavior patterns of a set of sleep events of the target subject based on the initial sensor data. For example, the system may identify baselines for a set of sleep events, such as a duration of bedtime preparation. The system generates a recommendation for achieving a target outcome for the target subject in the physical environment. For example, the system notes a noise level of activities outside of the bedroom and determines a causal relationship with target subject arousal. The system will then provide a recommendation of reducing a noise level of activities outside of the bedroom. Because the other sleep systems only use images for sensor data and do not provide detailed recommendations, they are not sufficiently personalized and do not achieve a target outcome. Moreover, image-based sensor data may be undesirable because of privacy concerns including medical concerns and because image-based sensor data requires sufficient light in the room, which can interfere with sleep.

In some embodiments, the behavior patterns are determined using a machine-learning model that is trained with training data that includes labels for each type of sleep event. This advantageously allows the system to provide insight that would otherwise be unavailable. In some embodiments, it is determined whether a recommendation was followed and, if so, whether the recommendation was successful in achieving the target outcome. The system may receive subsequent sensor data and generate updated behavior patterns, as well as an updated target outcome. As a result of receiving this feedback and modifying the behavior patterns accordingly, the system provides support over a sustained period to achieve target outcomes for the current age and all ages. Lastly, in addition to serving target subjects of all ages, the specification describes a system that is useful for target subjects with a variety of health conditions including special needs, mental health struggles, athletes, and people with medical issues.

### BRIEF DESCRIPTION OF THE DRAWINGS

FIG. 1 is a block diagram of an example network environment, according to some embodiments described herein.

FIG. 2 is a block diagram of an example flow of sensor data from a physical environment that is detected by a sleep hub and transmitted to a computing device for analysis, according to some embodiments described herein.

FIG. 3 is a block diagram of an example computing device, according to some embodiments described herein.

FIG. 4 is a block diagram of an example flow of sensor data from a physical environment to a thermal image sensor to a determination of patterns, according to some embodiments described herein.

FIG. 5 is a block diagram of an example flow of sensor data from a physical environment to a sound sensor to a determination of patterns, according to some embodiments described herein.

FIG. 6 is a block diagram of another example flow of how sound sensor data is used to determine patterns, according to some embodiments described herein.

FIG. 7 is a block diagram of an example flow of sensor data from a physical environment to a motion sensor to a determination of patterns, according to some embodiments described herein.

FIG. 8 is a block diagram of another example flow of how motion sensor data is used to determine patterns, according to some embodiments described herein.

FIG. 9 is a block diagram of an example flow of how initial sensor data is used to determine behavioral patterns, according to some embodiments described herein.

FIG. 10A illustrates an example user interface for configuring sleep preferences, according to some embodiments described herein.

FIG. 10B illustrates an example user interface for providing recommendations to achieve targeted behaviors, according to some embodiments described herein.

FIG. 10C illustrates an example user interface for providing a health check-in to a user, according to some embodiments described herein.

FIG. 10D illustrates an example user interface for providing an incentive for following the recommendation, according to some embodiments described herein.

FIG. 10E illustrates an example user interface for providing a sleep graph of a target subject, according to some embodiments described herein.

FIG. 10F illustrates an example user interface that includes a checklist of the recommendations, according to some embodiments described herein.

FIG. 11A illustrates an example graph of bedtime preparation duration as a function of nights based on a baseline stage, a stage when a recommendation is being followed, and a stage when a new recommendation is being followed, according to some embodiments described herein.

FIG. 11B illustrates an example graph of blocks where target outcomes are being achieved or not achieved during the stages illustrated in FIG. 11A, according to some embodiments.

FIG. 12 is a flow diagram illustrating azo example method to provide a recommendation to achieve a target outcome, according to some embodiments described herein.

### DETAILED DESCRIPTION

Network Environment 100

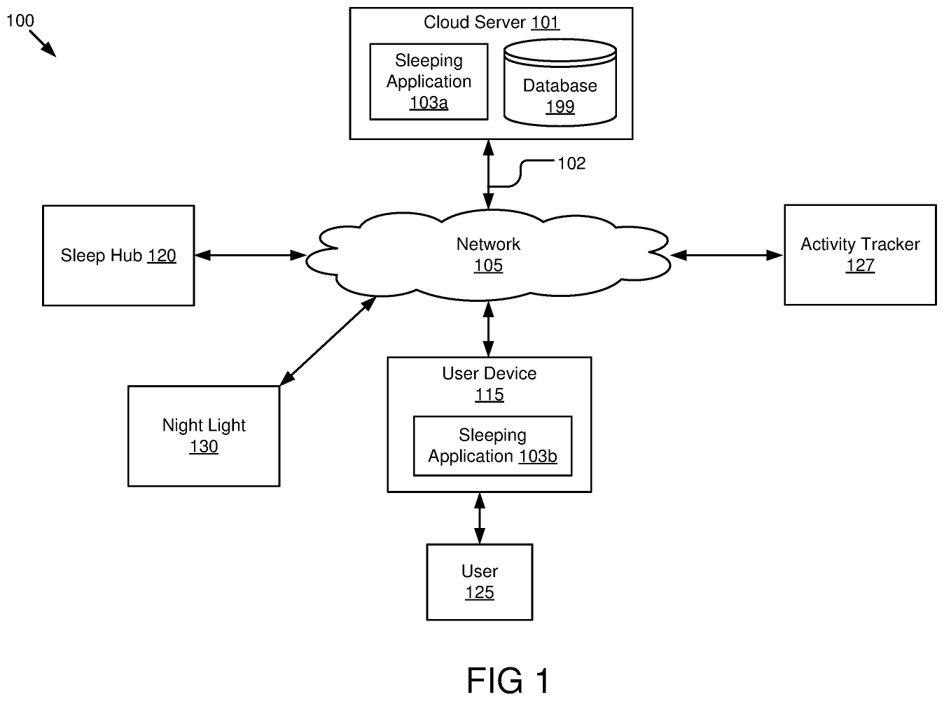

FIG. 1 illustrates a block diagram of an example network environment 100. In some embodiments, the network environment 100 includes a cloud server 101, a user device 115, a sleep hub 120, an activity tracker 127, a night light 130, and a network 105. User 125 may be associated with user device 115. In some embodiments, the environment 100 may include other servers or devices not shown in FIG. 1 or other entities illustrated in FIG. 1, such as the activity tracker, may not be included.

The cloud server 101 may include a processor, a memory, and network communication hardware. In some embodiments, the cloud server 101 is a hardware server. The cloud server 101 is communicatively coupled to the network via a wired connection, such as Ethernet, coaxial cable, fiber-optic cable, etc., or a wireless connection, such as Wi-Fi®, Bluetooth®, or other wireless technology. In some embodiments, the cloud server 101 sends and receives data to and from one or more of the user device 115, the sleep hub 120, and the activity tracker 127 via the network 105.

The cloud server 101 may include a sleeping application 103a and a database 199. In FIG. 1 and the remaining figures, a letter after a reference number, e.g., “103a,” represents a reference to the element having that particular reference number. A reference number in the text without a following letter, e.g., “103,” represents a general reference to embodiments of the element bearing that reference number.

The sleeping application 103a may include code and routines operable to receive initial sensor data from the sleep hub 120, the activity tracker 127, and/or the user device 115; determine behavior patterns of a set of sleep events of a target subject based on the initial sensor data; generate a recommendation based on the behavior patterns to achieve a target outcome for a target subject in the physical environment; and provide the recommendation. The target outcome may be provided by a user 125 of the user device 115 or determined based on comparing the behavior patterns to expected behavior for a target subject with similar attributes. For example, a user may be a parent that specifies that if a child rouses in the middle of the night, the child is able to put themselves back to sleep, which results in the sleeping application defining the target outcome as a child that puts themselves back to sleep. In another example, the sleeping application 103a may determine, by comparing the behavior patterns to expected behavior for a 53-year old man, that the target subject is getting two hours less than expected amount of sleep and, as a result, defines the target outcome as getting two more hours of sleep each night.

In some embodiments, the sleeping application 103a may be implemented using hardware including a central processing unit (CPU), a field-programmable gate array (FPGA), an application-specific integrated circuit (ASIC), any other type of processor, or a combination thereof. In some embodiments, the sleeping application 103a may be implemented using a combination of hardware and software.

The database 199 may store information associated with a person. For example, the database 199 may store both the initial sensor data and the subsequent sensor data, the behavior patterns, user preferences, etc.

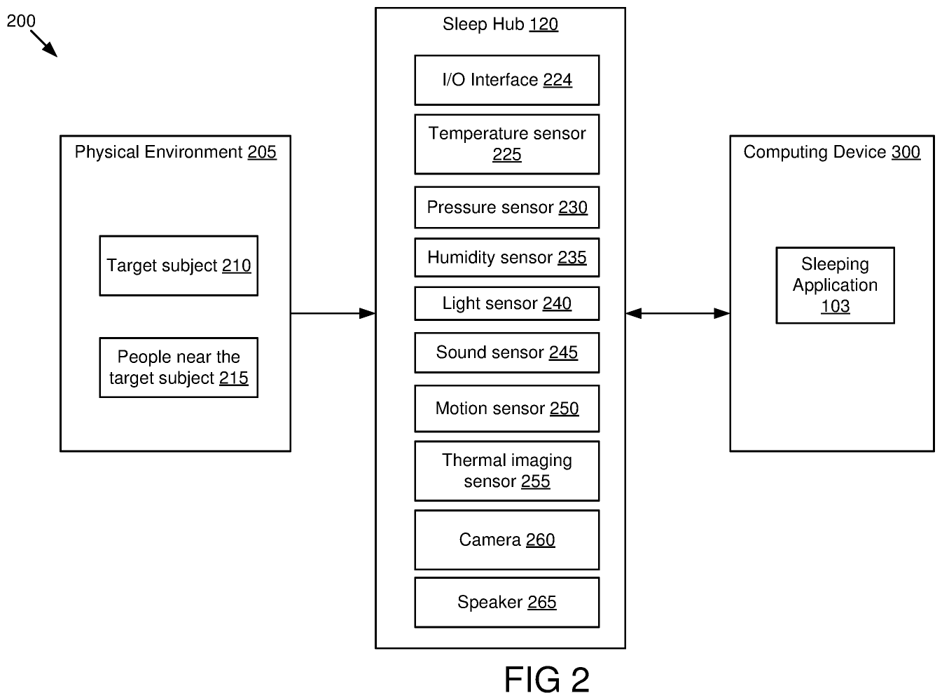

The sleep hub 120 may be a computing device that includes memory, a hardware processor, a sensor set, a speaker, and an input/output (I/O) interface. The components of the sleep hub 120 are described in greater detail below with reference to FIG. 2. The sensors generate sensor data and transmit the sensor activity to the sleeping application 103 stored on the cloud server 101 and/or the user device 115. For example, a sensor may detect activity within a room where a person is sleeping, a change in the lights in the room, a change in humidity in the room, etc. In some embodiments, multiple rooms contain a respective sleep hub 120 and coordinate sensor data. For example, conventional wisdom holds that if a person has trouble sleeping, they should not stay in the bed because the bed should only be associated with sleep. As a result, a hub in a bedroom could detect the person's behavior while trying to sleep and a hub in a living room could detect the person trying to read when they have trouble sleeping.

In some embodiments, the speaker generates white noise or audio associated with a sleep assistant that helps a person with sleep-related activities. The white noise may use sound attenuating technology to determine the volume based on the size of the physical environment, the proximity of the product from the bed, the amount of sound absorbing fabric in the room, etc. In some embodiments, an I/O interface is operable to provide instructions to the speaker. For example, a user may use the sleeping application 103b on the user device 115 to activate the white noise machine or another sleep assistant function. The sleeping application 103b on the user device 115 may transmit instructions to the sleep hub 120 to activate the speakers to carry out the instructions to produce white noise or sleep assistant prompts. In some embodiments, the sleeping application 103b includes recorded voices provided by a user, such as a lullaby sung by a grandparent or a story read by a parent.

The night light 130 may be a computing device that includes memory, a hardware processor, and a light. In some embodiments, the night light 130 receives a notification from the sleep hub 120 that a someone is detected in the room with the sleep hub 120. In response, the night light 130 may switch from a dark mode to emitting a light so that the person entering the room can find the night light 130. In some embodiments, the night light 130 may switch from a dark mode to emitting a light when a person picks up the night light 130 and/or detaches the night light 130 from a cradle.

The light may be designed to be bright enough that a parent can use it to change diapers, but not so bright that it interferes with a baby's ability to sleep. In some embodiments, the night light 130 is portable. In some embodiments, when the child is older, the night light 130 may be configured to emanate a soft unstimulating glow. The soft unstimulating glow may be used, for example, to help a child with nighttime toilet training.

The activity tracker 127 may be a computing device that includes memory, a hardware processor, a display screen, and sensors. For example, the activity tracker 127 may have an onboard global positioning system (GPS), an altimeter, a heartrate monitor, etc. that are operable to track runs, cardiovascular activities, hikes, biking, etc. In some embodiments, the activity tracker 127 uses a motion sensor to detect when the user is sleeping. The activity tracker 127 is connected to the network 105 and transmits sensor data to the sleeping application 103.

The user device 115 may be a computing device that includes a memory and a hardware processor. For example, the user device 115 may include a desktop computer, a mobile device, a tablet computer, a mobile telephone, a wearable device, a head-mounted display, a mobile email device, a portable game player, a portable music player, a reader device, or another electronic device capable of accessing a network 105.

In the illustrated implementation, user device 115a is coupled to the network 105 by a wired connection, such as Ethernet, coaxial cable, fiber-optic cable, etc., or wireless connections, such as Wi-Fi®, Bluetooth®. The sleeping application 103 may be stored as a sleeping application 103b on the user device 115. While only one user device 115 is illustrated, in some embodiments, user device 115a and user device 115n are part of the network environment 100. For example, the sleep hub 120 may be used to analyze the behavior patterns of a baby, user device 115a is used by a first parent (user 125a), and a user device 115b is used by a second parent (user 125b).

In some embodiments, the sleeping application 103b receives sensor data from other applications on the user device 115. For example, the user may use a meditation application to try and get more restful sleep or fall back asleep after an arousal. The sleeping application 103b integrates the sensor data from the other applications on the user device 115.

Sleep Hub 120

FIG. 2 is a block diagram of an example flow of sensor data from a physical environment 205 that is detected by a sleep hub 120 and transmitted to a computing device 300 for analysis. In some embodiments, the physical environment 205 includes a target subject 210 and people near the target subject 215. For example, the physical environment 205 may be a house, the target subject 210 may be a baby (an infant, a toddler, a child, a teenager, etc.) and the people near the target subject 215 may be parents in the room with the baby, people outside the room that are loud enough that their actions are detected as sensor data, etc. In another embodiment, the physical environment 205 may be an apartment building, the target subject 210 may be an adult with insomnia, and the people near the target subject 215 may be people in apartments that are next to the adult's apartment.

The sleep hub 120 may include an I/O interface 224, a sensor set with one or more of a temperature sensor 225, a pressure sensor 230, a humidity sensor 235, a light sensor 240, a sound sensor 245, a motion sensor 250, a thermal imaging sensor 255, a camera 260, and a speaker 265.

The I/O interface 224 can provide functions to enable interfacing between the sleep hub 120 and the computing device 300, and also with components within the sleep hub 120. For example, the I/O interface 224 receives sensor data from any of the sensors in the sensor set and transmits the sensor data to the computing device 300. In another example, the I/O interface 224 receives instructions from the computing device 300, such as to activate the speaker 265 to play white noise, and the I/O interface 224 carries out the instructions.

The temperature sensor 225 includes hardware that senses a temperature in the physical environment 205. The pressure sensor 230 includes hardware that senses a pressure in the physical environment 205 or a specific pressure in a particular location. For example, the pressure sensor 230 may be underneath a baby's crib and is used to detect a change in pressure that would indicate that the baby left the crib. The humidity sensor 235 includes hardware that senses a humidity in the physical environment 205. The light sensor 240 includes hardware that senses a level of light in the physical environment 205. The sound sensor 245 includes hardware that senses a level of sound in the physical environment 205 and converts the sound level to decibels. In some embodiments, the sound sensor 245 includes a microphone. In some embodiments, the sounds are recorded and stored as audio files (subject to user consent to store the audio files).

The motion sensor 250 includes hardware that senses motion in the physical environment 205. For example, the motion sensor 250 may include one or more of a passive infrared sensor that detects body head by looking for changes in temperature, a microwave sensor that sends out microwave pulses and measures the reflects off of moving objects, an area reflective sensor that emits infrared rays from a light-emitting diode (LED) and uses the reflection of the rays to measure the distance to the target subject or object, an ultrasonic motion sensor that measures the reflections off of moving objects via pulses of ultrasonic waves, and a vibration motion sensor that detects small vibrations that people cause when they move through a room.

The thermal imaging sensor 255 includes hardware that uses infrared (IR) radiation to sense people and objects in a physical environment. For example, the thermal imaging sensor 255 may include an IR camera that uses IR radiation or thermal imaging to track the locations of the people and objects in a room.

The camera 260 includes hardware that captures images and/or video of the physical environment 205. In some embodiments, the camera 260 receives a detection of motion from the motion sensor 250 via the I/O interface 224 that a person is detected in the room and captures images and/or video of the physical environment 205 in response to the motion being detected. In some embodiments, the camera 260 captures images and/or video of the physical environment 250 when the light level in the room exceeds a threshold amount.

Computing Device 300 Example

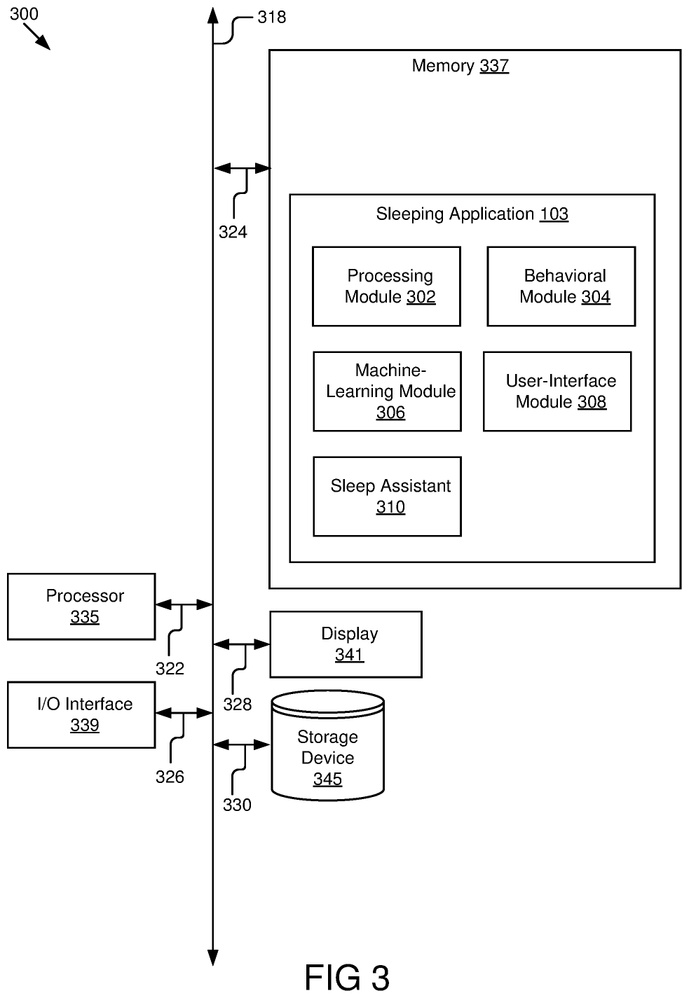

FIG. 3 is a block diagram of an example computing device 300 that may be used to implement one or more features described herein. Computing device 300 can be any suitable computer system, server, or other electronic or hardware device. In one example, computing device 300 is a user device 115 used to implement the sleeping application 103. In another example, computing device 300 is the cloud server 101. In yet another example, the sleeping application 103 is in part on the user device 115 and in part on the cloud server 101.

In some embodiments, computing device 300 includes a processor 335, a memory 337, a I/O interface 339, a display 341, and a storage device 345, all coupled via a bus 318. The processor 335 may be coupled to the bus 318 via signal line 322, the memory 337 may be coupled to the bus 318 via signal line 324, the I/O interface 339 may be coupled to the bus 318 via signal line 326, the display 341 may be coupled to the bus 318 via signal line 328, and the storage device 345 may be coupled to the bus 318 via signal line 330. Certain components may be added or removed, depending on the type of computing device 300 that is applicable. For example, where the computing device 300 is a cloud server 101, the computing device 300 may not include a display 341.

The processor 335 includes an arithmetic logic unit, a microprocessor, a general-purpose controller or some other processor array to perform computations and provide instructions to a display device. Processor 335 processes data and may include various computing architectures including a complex instruction set computer (CISC) architecture, a reduced instruction set computer (RISC) architecture, or an architecture implementing a combination of instruction sets. Although FIG. 2 includes a single processor 335, multiple processors 335 may be included. Other processors, operating systems, sensors, displays and physical configurations may be part of the computing device 200.

Memory 337 is typically provided in computing device 300 for access by the processor 335, and may be any suitable processor-readable storage medium, such as random access memory (RAM), read-only memory (ROM), Electrical Erasable Read-only Memory (EEPROM), Flash memory, etc., suitable for storing instructions for execution by the processor or sets of processors, and located separate from processor 335 and/or integrated therewith. Memory 337 can store software operating on the computing device 300 by the processor 335, including a sleeping application 103.

The memory 337 stores instructions that may be executed by the processor 335 and/or data. The instructions may include code for performing the techniques described herein. The memory 337 may be a dynamic random access memory (DRAM) device, a static RAM, or some other memory device. In some implementations, the memory 337 also includes a non-volatile memory, such as a (SRAM) device or flash memory, or similar permanent storage device and media including a hard disk drive, a floppy disk drive, a compact disc read only memory (CD-ROM) device, a DVD-ROM device, a DVD-RAM device, a DVD-RW device, a flash memory device, or some other mass storage device for storing information on a more permanent basis. The memory 337 includes code and routines operable to execute the sleeping application 103, which is described in greater detail below.

I/O interface 339 can provide functions to enable interfacing the computing device 300 with other systems and devices. Interfaced devices can be included as part of the computing device 300 or can be separate and communicate with the computing device 300. For example, network communication devices, storage devices (e.g., memory 337 and/or database 199), and input/output devices can communicate via I/O interface 339. In some embodiments, the I/O interface 339 can connect to interface devices such as input devices (keyboard, pointing device, touchscreen, microphone, camera, scanner, sensors, etc.) and/or output devices (display devices, speaker devices, printers, monitors, etc.). For example, when a user provides touch input, I/O interface 339 transmits the data to the sleeping application 103.

Some examples of interfaced devices that can connect to I/O interface 339 can include a display 341 that can be used to display content, e.g., a user interface generated by the sleeping application 103 as described herein, and to receive touch (or gesture) input from a user. For example, display 341 may be utilized to display a user interface that receives user input from a user.

Display 341 can include any suitable display device such as a liquid crystal display (LCD), light emitting diode (LED), or plasma display screen, cathode ray tube (CRT), television, monitor, touchscreen, three-dimensional display screen, or other visual display device. For example, display 341 can be a flat display screen provided on a mobile device, multiple display screens embedded in a glasses form factor or headset device, or a monitor screen for a computer device.

The storage device 345 stores data related to the sleeping application 103. For example, the storage device 345 may store both the initial sensor data and the subsequent sensor data, the behavior patterns, user preferences, etc. In embodiments where the sleeping application 103 is part of the cloud server 101, the storage device 345 is the same as the database 199 in FIG. 1.

Sleeping Application 103

FIG. 3 illustrates an example sleeping application 103 that includes one or more of a processing module 302, a behavioral module 304, a machine-learning module 306, a user-interface module 308, and a sleep assistant 310.

The processing module 302 receives sensor data from the sleep hub 120 and organizes the sensor data. In some embodiments, the processing module 302 includes a set of instructions executable by the processor 335 to process the sensor data. In some embodiments, the processing module 302 is stored in the memory 337 of the computing device 300 and can be accessible and executable by the processor 335.

In some embodiments, the processing module 302 organizes the sensor data into an understandable behavior or metric. For example, the processing module 302 may receive sensor data from a thermal image sensor, determine an identity of the people, and track their movement. In another example, the processing module 302 receives sensor data from the light sensor 240 and determines a brightness in the room.

In some embodiments, the processing module 302 applies thresholds to the organized sensor data to determine whether the organized sensor data is within an acceptable level. For example, the processing module 302 may retrieve a temperature threshold from the storage device 345 that states that the temperature should be between 68 degrees and 72 degrees. If the temperature falls below or exceeds the temperature threshold, the processing module 302 may instruct the user-interface module 308 to send an alert to a user to warn the user about the temperature. In another example, the processing module 302 may apply a brightness threshold to the room where the brightness threshold is different depending on whether the target subject is napping or sleeping (e.g., the brightness threshold is much lower for sleeping for napping for a baby, but the brightness threshold may be different for a person that works the night shift and sleeps at a different time of the day).

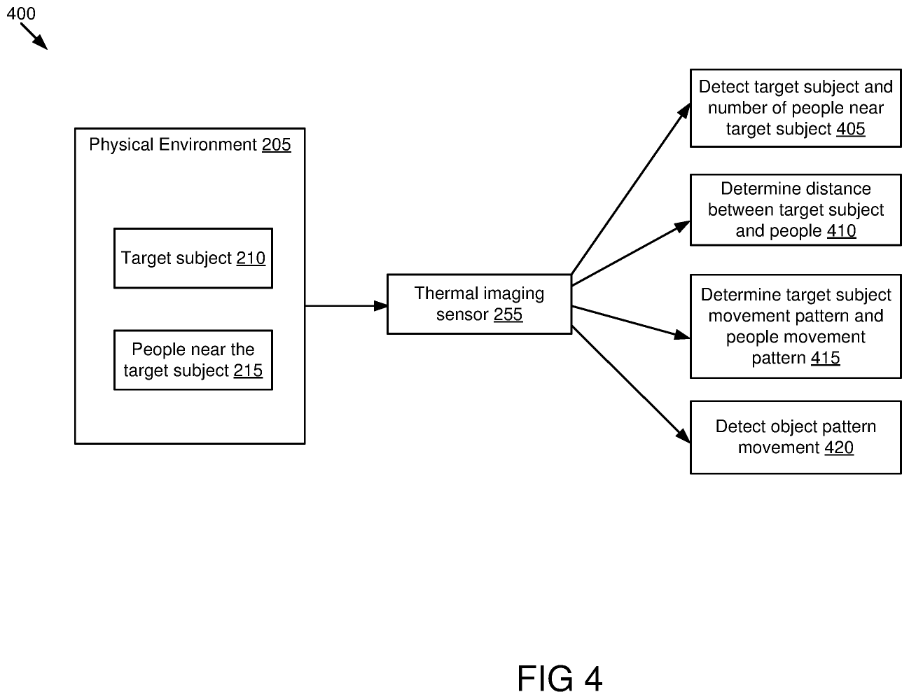

FIG. 4 illustrates a block diagram 400 of an example flow of sensor data from a physical environment 215 to a thermal sensor to a determination of information. The thermal imaging sensor 255 generates thermal images where the target subject 210 and people near the target subject 215 are present in the physical environment 205. The processing module 302 uses the thermal images to detect 405 the target subject and a number of people near the target subject, determine 410 a distance between the target subject and the people, determine 415 a target subject movement pattern and a people movement pattern, and detect 420 object pattern movement. For example, where the target subject 210 is an adult, the behavioral module 304 may determine a behavioral pattern based on a target subject movement pattern that shows that the adult becomes restless in bed and starts tossing and turning when another adult enters the room, especially when the other adult comes close to the adult. In another example where the target subject 210 is an adult, the target subject may consistently experience an arousal when the target subject sleeps next to another adult, but sleeps more soundly when sleeping alone.

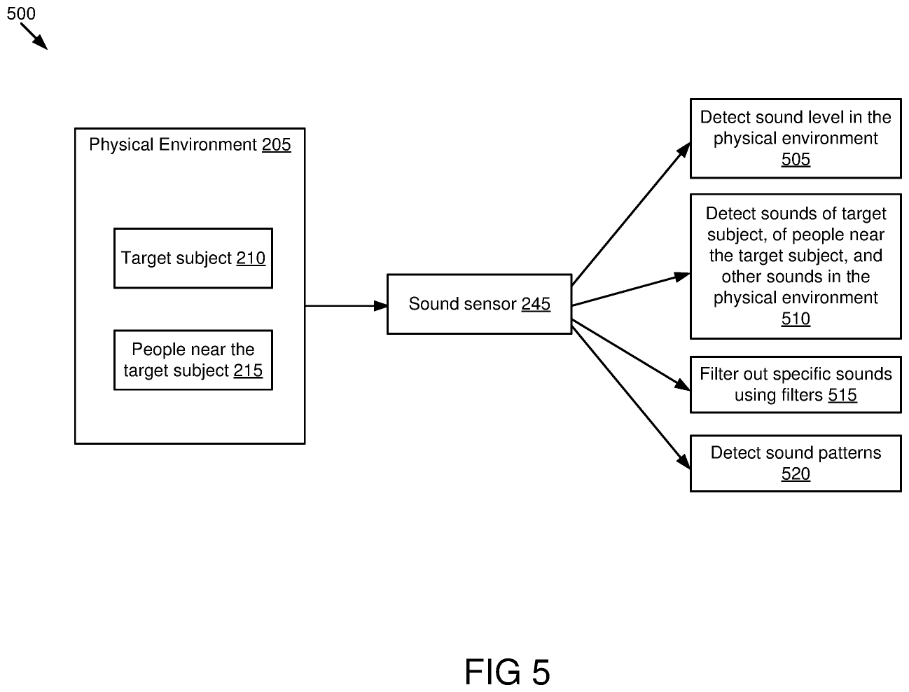

FIG. 5 is a block diagram 500 of an example flow of sensor data from a physical environment 205 to a sound sensor 245 to a determination of patterns. The sound sensor 245 generates sensor data based on sounds from the target subject 210, sounds from people near the target subject 215, sounds that permeate the physical environment 205 from other locations. etc. The processing module 302 detects 505 a sound level in the physical environment. The processing module 302 identifies who makes a sound (e.g., parent, child, dog, etc.) and a purpose of the noise (e.g., from reading a story, singing, comforting sounds, a crying baby versus a babbling baby, a sound of an electronic device including a television or music from another room, sound from regular domestic activities, etc.). For example, the processing module 302 receives initial sensor data from a sound sensor 245 that includes a microphone, applies sound processing techniques to determine the sound level, and determines that at 8:02 pm the decibel level in the room was 25 dB, at 8:03 pm the decibel level increased to 60 dB from a baby gently crying, at 8:04 pm the decibel level decreased to 40 dB as the baby started to settle, etc.

The processing module 302 detects 510 sounds of the target subject, people near the target subject, and other sounds in the physical environment. In some embodiments, the sound sensor 245 includes a microphone that can discriminate between different types of sound. For example, the processing module 302 may determine that the increase in sound in the physical environment 205 came from a noise that originated outside the room. In some embodiments, the processing module 302 detects the sounds associated with the target subject, the people near the target, and the other sounds by capturing audio and running audio processing and machine-learning algorithms on the captured audio. In some embodiments, the processing module 302 determines a distance between the target subject and other people in the room by applying audio processing techniques.

The processing module 302 filters 515 out specific sounds using filters. For example, the processing module 302 applies a filter for the hum produced by electronic devices. The processing module 302 detects 520 sound patterns. For example, the processing module 302 may identify different noises and variations in the sound levels of each of the noises as a function of time.

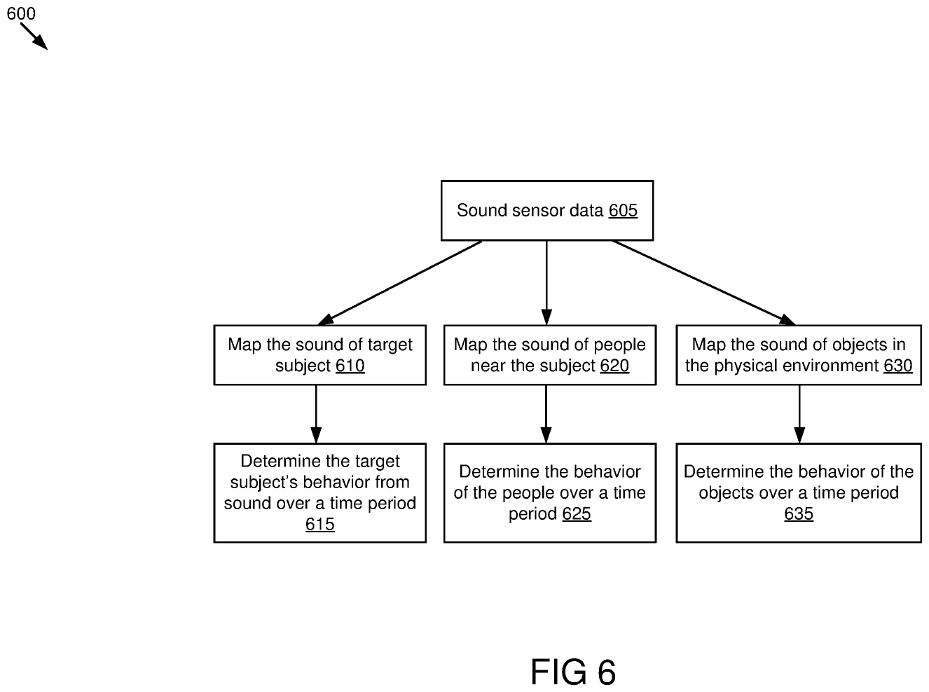

FIG. 6 is a block diagram 600 of another example flow of how sound sensor data is used to determine patterns, according to some embodiments described herein. In this example, the processing module 302 uses the sound sensor data 605 to map 610 the sound of the target subject and determine 615 the target subject's behavior from sounds over a time period. For example, the target subject produced different decibel levels of sound over a time period. The processing module 302 uses the sound sensor data 605 to map 620 the sound of people near the subject and determine 625 the behavior of the people over a time period. The processing module 302 uses the sound sensor data 605 to map 630 the sound of objects in the physical environment and determine 635 the behavior of the objects over a time period.

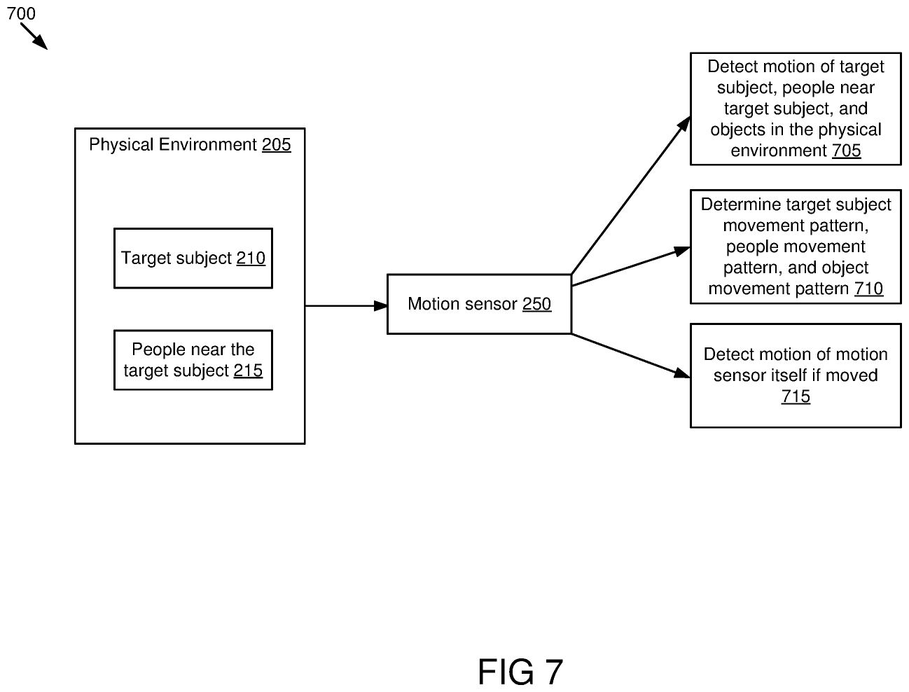

FIG. 7 is a block diagram 700 of an example flow of sensor data from a physical environment 205 to a motion sensor to a determination of patterns, according to some embodiments described herein. The motion sensor 250 generates sensor data based on motion from the target subject 210, motion from people near the target subject 215, motion of objects in the physical environment 205, and motion from the motion sensor 250. The processing module 302 detects 705 motion of the target subject, people near the target subject, and objects in the physical environment. The processing module 302 determines 710 a target subject movement pattern, a people movement pattern, and an object movement pattern. The processing module 302 detects 715 motion of the motion sensor itself if it is moved.

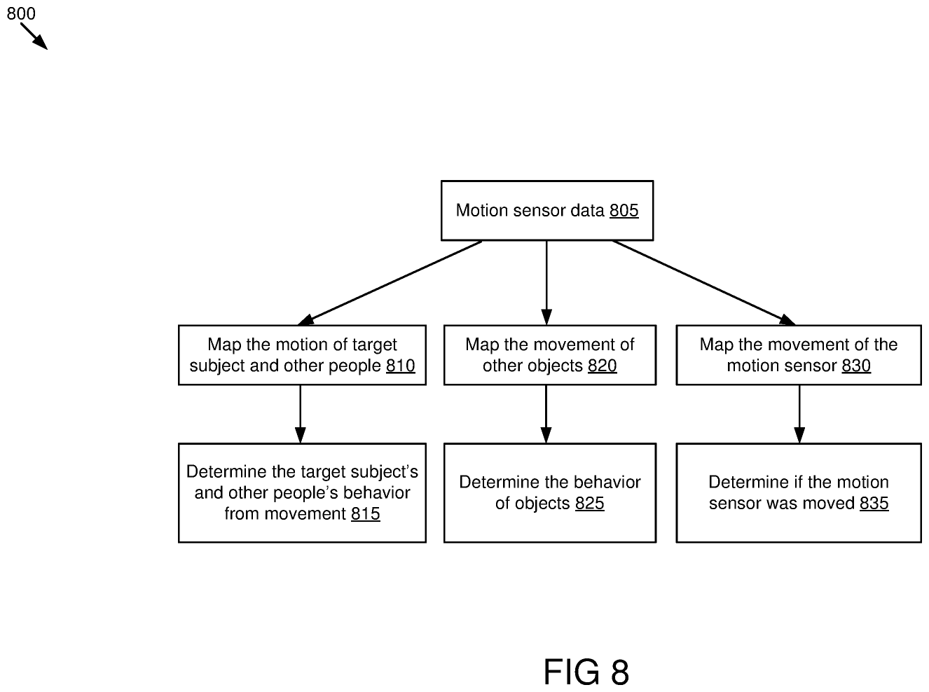

FIG. 8 is a block diagram 800 of another example flow of how motion sensor data is used to determine patterns, according to some embodiments described herein. In this example, the processing module 302 uses the motion sensor data 805 to map 810 the motion of the target subject and other people and determine 815 the target subject's and other people's behavior from movements. For example, the processing module 302 may determine that the target subject sat up in their sleep and that another person got into bed with the target subject. The processing module 302 uses the motion sensor data 805 to map 820 the movement of the other objects and determine 825 the behavior of the objects. For example, a toy in the room vibrates and spins around or a pacifier falls from the crib to the floor. The processing module 302 maps 830 the movement of the motion sensor and determines 835 if the motion sensor was moved.

The behavioral module 304 determines behavior patterns of a set of sleep events. In some embodiments, the behavioral module 304 includes a set of instructions executable by the processor 335 to determine the behavior patterns. In some embodiments, the behavioral module 304 is stored in the memory 337 of the computing device 300 and can be accessible and executable by the processor 335.

In some embodiments, the behavioral module 304 distinguishes between initial sensor data received for a time period and subsequent sensor data received from the processing module 302. For example, the behavioral module 304 may collect initial sensor data for 24 hours, two days, a week, a month, etc. to determine a baseline of behavior patterns. The subsequent sensor data is received after the behavior patterns are determined in order to assess whether a target outcome is being achieved.

The behavioral module 304 determines a baseline of behavior patterns of a set of sleep events of a target subject based on initial sensor data. The set of sleep events may include a bedtime preparation, a nighttime arousal with a sleep-cycle change, a nighttime arousal with a parent, a target subject leaves the physical environment, a nighttime over, a naptime preparation, a naptime arousal with a sleep-cycle change, a naptime arousal with a parent, a naptime over, and sleeping. The behavioral module 304 may determine attributes associated with a sleep event from a set of sleep events, such as times that the target subject started and stopped each of the sleep events. The behavioral module 304 also determines, as part of the baseline of behavior patterns, actions relating to the set of sleep events. For example, the behavioral module 304 determines that a parent will respond to a nighttime arousal with a parent by entering the physical environment and attempting to soothe the target subject back to sleep.

The attributes for bedtime preparation may be a first flutter of movement and vocalizations in the physical environment, lights on in the physical environment, or more than one person present in the physical environment at the onset of bedtime. For example, after a bedtime preparation period of 45 minutes, a particular baby may then spend a total duration of 10.2 hours in their room at night based on an average as determined from the initial sensor data. In some embodiments, the behavioral module 304 applies an offset to one or more of the sleep events before defining a sleep event as occurring. For example, an end of the bedtime preparation may not occur unless there are 15 minutes where the light is dark, the sound machine is on (if being used), and the movement is minimal (if the target subject is the only one in the room). If, for example, movement occurs after the 15 minutes, the behavioral module 304 categorizes the movement as part of a nighttime arousal. In some embodiments, the behavioral module 304 also tracks whether a parent stays in the physical environment after the target subject falls asleep. If the parent stays in the room, the behavioral module 304 may determine that the end of the bedtime preparation does not occur unless there are 15 minutes where the light is dark and the target subject does not interact with a parent.

The attributes for nighttime arousal may be a short period of time when a target subject rouses in the middle of the night and can include movement in the bed and vocalizations (e.g., baby crying, person talking in their sleep, coughing, clearing throat, etc.). In some embodiments, the nighttime arousal may include a sleep-cycle change, such as a target subject opening their eyes, moving around and going back to bed, getting up to use the bathroom and returning to bed, etc.

Nighttime arousal with a parent includes a period of interaction between the parent and target subject (e.g., baby, child, etc.). The nighttime arousal with a parent may include vocalizations from both the parent and the person, movement from either of the parent and the person (e.g., a parent entering the physical environment or getting out of bed if the parent sleeps in the room with the target subject), an increase in light saturation in the physical environment, a feeding, a diaper change, etc. The nighttime arousal with a parent ends when the parent leaves the room and the target subject is quiet and not moving.

The attributes for a target subject leaving the physical environment may include any time that the target subject leaves the physical environment and subsequently returns to complete sleep.

The attributes for nighttime being over may include an increase in light saturation, increased movement and vocalizations (e.g., talking, a diaper change, feeding, etc.) that do not end with a parent returning the target subject to the bed, laying down in a bed in the physical environment, or removing the target subject from the physical environment and not returning to the physical environment for a period of time.

The attributes for a naptime preparation may include a time between the first flutter of movement and vocalizations in the physical environment at the onset of a desired naptime. In some embodiments, the behavioral module 304 distinguishes between naptime and nighttime based on a time of day. In some embodiments, the offset is defined as a predetermined amount of time (e.g., fifteen minutes) where the light is dark, the sound machine is on (if being used), and the movement is minimal.

The attributes for a naptime arousal may be a short period of time when a target subject rouses in the middle of the night and can include movement in the bed and vocalizations. In some embodiments, the naptime arousal includes a sleep-cycle change.

The attributes for a naptime arousal with a parent may include vocalizations from both the parent and the person, movement from either of the parent and the person, an increase in light saturation in the physical environment, a feeding, a diaper change, etc. The naptime arousal with a parent ends when the parent leaves the room and the target subject is quiet and not moving.

The attributes for a naptime being over may include an increase in light saturation, increased movement and vocalizations, and the target subject leaves the room for a predetermined amount of time (e.g., 15 minutes) and does not return.

The attributes for sleeping may include a period of time when the room is quiet, minimal movement from the target subject and anyone else in the physical environment, and minimal vocalizations as defined by a threshold range of decibel levels. If, for example, a child sleeps from 10 pm-2 am and 3 am-7 am, the sleep is logged as eight hours because it does not include the nighttime arousal from 2 am-3 am.

One advantage of defining a sleep event based on a fixed set of attributes is that the behavioral module 304 generates a consistent way to measure sleep events and determine whether there is a correlation or causation between activities and the different types of sleep events. For example, the behavioral module 304 defines sleeping as occurring when the room is quiet, minimal movement is occurring from anyone in the room, and minimal vocalizations occur. Conversely, one parent may define arousal from sleep as occurring even if a baby starts talking because the parent believes the talking is minimal. As a result, this parent may decide that the baby only woke up two times in a night when the behavioral module 304 identified four arousals. Thus, the behavioral module 304 recognizes a problem that is more likely to interfere with sleep than the parent. In a second example, a second parent may decide that the exact same behavior presented to the first parent constitutes six arousals in a night because the second parent may interpret slight mumbling as an arousal. As a result, the behavioral module 304 identifies that the problem is not as severe as the parent believes because the parent does not recognize that a baby may still be sleeping while talking. Furthermore, the behavioral module 304 measures the impact of an intervention so parents can easily determine if sleep metrics are improving.

The behavioral module 304 may determine the behavior patterns based on the initial sensor data from one of the sensors or by combining the initial sensor data from multiple sensors. For example, the behavioral module 304 may determine that as a result of a noise from an object detected by the sound sensor 245, a woman sleeping in the bed started to move as detected by the motion sensor 250, and that the motion was enough to determine that a nighttime arousal event occurred. In another example, the behavioral module 304 may determine that when an adult with insomnia played music that is set on a timer and the adult fell asleep while the music was playing, cessation of the music results in a consistent pattern of arousals each night where the adult had trouble falling back asleep for three hours after arousal. In yet another example, the behavioral module 304 may determine that an adult that takes medication during the night for a medical issue (e.g., cancer), may have trouble falling back asleep after arousal.

In some embodiments, the initial sensor data is received from a combination of the set of sensors that are part of the sleep hub 120, the activity tracker 127, and a user device 115. The behavioral module 304 may determine the behavior patterns based on the initial sensor data from the different sources. For example, the behavioral module 304 may determine that a mother came into the physical environment during a nighttime sound of a baby crying because the thermal imaging sensor 255 detected an adult coming into the room, a motion sensor 250 included a vibration sensor that identified the vibrations with enough precision that the processing module 302 identified the vibrations as belonging to a woman, and the initial sensor data from the activity tracker 127 confirmed that the mother was walking at the time that a woman entered the physical environment with the baby.

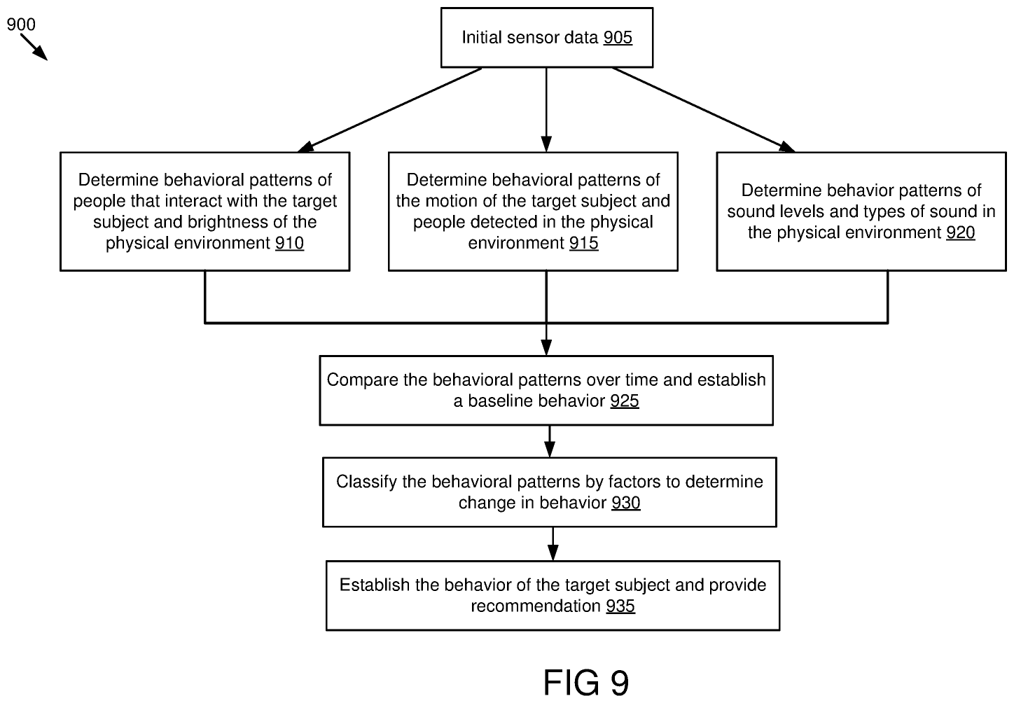

FIG. 9 is a block diagram 900 of an example flow of how initial sensor data is used to determine behavioral patterns. In this example, the initial sensor data 905 is received from a light sensor 240, a motion sensor 250, and a sound sensor 245, but other combinations and additional types of initial sensor data may be used. The behavioral module 34 receives the initial sensor data 905 as organized by the processing module 302. The behavioral module 304 determines 910 behavioral patterns of people that interact with the target subject and brightness of the physical environment, determines 915 the behavioral patterns of the motion of the target subject and people detected in the physical environment, and determines 920 the behavioral patterns of sound levels and types of sound in the physical environment. For example, the behavioral module 304 may determine a pattern of a parent sitting in a rocking chair or on a couch, a target subject lying down, a parent changing a diaper, a parent putting the baby in the crib, the parent taking the baby out of the crib, etc. The behavioral module 304 compares 925 the behavioral patterns over time and establishes a baseline behavior. For example, the behavioral module 304 may identify a baseline for sleep-onset delay where sleep-onset delay is defined as the amount of time that elapses from when a parent leaves the room to when the child falls asleep. The behavioral module 304 classifies 930 the behavioral patterns by factors to determine change in behavior over a time period (e.g., throughout the week). For example, the start of the bedtime preparation may be later on the weekends, a nap may be skipped on a busy day, sleeping may be longer on a weekday, etc. Establishing the baseline is helpful for determining an expected amount of activity. For example, a target subject may regularly get up in the middle of the night to use the bathroom and this may not be something that can be changed with a behavior outcome.

Once the behavioral module 304 establishes the behavior patterns, the behavioral module 304 generates a recommendation for achieving a target outcome based on the behavior patterns. In some embodiments, the target outcome is established by a user. For example, the user may state that a target outcome is a reduction of a sleep-onset delay to 30 minutes or less, elimination of sleep-interfering behavior, and achievement of 10.5 hours of uninterrupted sleep as indicated by the attributes for sleeping discussed above (e.g., minimal motion). In another example, the target outcome may be reduction of sleep-onset delay to 15 minutes or less, elimination of interfering behaviors, reduction of night and early arousals, achievement of 12 hours of independent sleep, elimination of parental presence at night, elimination of medication, and elimination of supplements.

In another example, the behavior module 304 analyzes the behavioral pattern of a target subject with autism spectrum disorder (ASD) to determine that the sleep-onset delay is 30 minutes to one hour and suggests reduction of the sleep-onset delay as a target outcome. The behavior module 304 also identifies a behavior pattern that the bedtime preparation includes the target subject changing into pajamas and watching television for 40 minutes, browsing the internet on a tablet, or playing loud games on the tablet. The behavior module 304 generates a recommendation that includes playing loud games on the tablet before changing into pajamas to create distance between a stimulating activity and the bedtime preparation.

In some embodiments, as discussed in greater detail below with reference to the user-interface module 308, the user may specify one or more target outcomes to be achieved. For example, the target outcome may suggest a target outcome that is common for a target subject of the particular person's age. In some embodiments, the behavioral module 304 may instruct the user interface module 308 to walk through a series of questions designed to identify the target outcome. For example, the user interface may include a section where a user can identify that the sleep problems relate to an inability to fall asleep, an inability to remain asleep, an inability to sleep for an age-appropriate length, etc. The user interface may then include options for further narrowing the sleep problems, such as whether the inability to remain asleep is evidenced by noise coming from the room, a child causing a parent to rouse, a child requiring that a parent be present for the child to fall back asleep, a child acting lethargic the next morning, etc.

In some embodiments, the target outcomes include target outcomes for a child and target outcomes for the parent. For example, if the child starts falling asleep faster, the parent may need reminders to take advantage of the extra sleep time and go to bed themselves. The recommendation may include a list of items that the user should continue to do and a list of items that are interfering with achievement of the target outcome. For example, the list of items that the user should continue to do may include the ideal temperature for the room, the ideal light level in the room, a recommendation to stop using devices with screens that emit blue lights because they interfere with production of melatonin. In another example, the list of items that the user should continue to do may include not checking on the baby after hearing the first signs of vocalizations, not reading bedtime stories in an animated voice, and not sleeping in the room with the baby after a nighttime arousal has occurred.

The behavioral module 304 establishes 935 the behavior of the target subject and provides a recommendation to a user. In some embodiments, the recommendation includes a report of the behavior patterns. For example, a target subject experiencing insomnia due to post-traumatic stress disorder may receive a report that identifies a correlation between certain types of sounds and nighttime arousal. In another example, the target subject may be an elderly person and a caregiver may receive a recommendation with a report of the elderly person's activities when they are trying to sleep.

The behavioral module 304 may generate a recommendation based on the behavioral patterns by identifying factors that contributed to behavior that interferes with the target outcome. For example, the behavioral patterns may include a baseline of a sleep where an adult slept through the night and an incident where the adult woke up. The behavioral module 304 may identify an incident where the adult woke up was precipitated by factors that were different than the baseline information, such as a temperature outside of the temperature threshold, a loud noise that was followed by the adult beginning to move, etc. or other factors, such as subsequent sensor data from an activity tracker 127 where the adult reported experiencing an unusual amount of stress.

In some embodiments, the behavioral module 304 identifies reinforcers for sleep problems and generates a recommendation that minimizes the reinforcers. For example, the behavioral module 304 may identify a behavior pattern where a child rouses on average four times a night and plays with toys in the bed. The behavioral module 304 generates a recommendation to minimize the reinforcers of “playing with toys” behavior by recommending a series of steps that will help guide the parent in removing the toys and books from the bed and will subsequently measure nighttime disruptions to determine if the intervention is working as expected.

In some embodiments, the behavioral module 304 assigns a recommendation score to each recommendation based on the likelihood of a recommendation to achieve the target outcome. For example, each recommendation may be scored on a scale from 1 to 100 where other scales are possible, such as from 0 to 1, a percentage, etc. In some embodiments, the behavioral module 304 generates a recommendation if there is more than a threshold likelihood that the recommendation with achieve the targeted behavior. For example, if a baseline sleep length is established with a set of conditions and changing the temperature results in a 75% chance that the sleep length is modified, the behavioral module 304 may assign a recommendation score for a recommendation to keep the temperature within a threshold range that was present for the baseline sleep length because the 75% exceeds a threshold value of 70%.

In some embodiments, the behavioral module 304 may receive subsequent sensor data as organized by the processing module 302; determine, based on a comparison of the subsequent sensor data to the behavior patterns, that an action is likely to precipitate a nighttime arousal or a naptime arousal; and provide a warning that the action is likely to precipitate the nighttime arousal or the naptime arousal. For example, the behavioral module 304 may identify that a parent is watching television loudly enough that the sound is permeating the physical environment where the baby is sleeping and it might cause a nighttime arousal because during establishment of the behavioral patterns the baby woke up when the same level of noise permeated the physical environment.

If the user follows the recommendation and the target outcome was achieved, the behavioral module 304 may use the subsequent sensor data to update the behavior patterns. For example, the behavioral module 304 may establish a new baseline for each of the set of sleeping events. In some embodiments, the behavioral module 304 updates the target outcome based on updating the behavior patterns. For example, the behavioral module 304 may automatically determine a new target outcome and suggest it to the user or the user-interface module 308 may ask the user if they want to establish a new target outcome based on the success of achieving the previous target outcome. For example, where the target outcome for a teenager is reduced delayed onset where the baseline behavior is a delayed onset of one hour, the behavioral module 304 may initially recommend pushing the teenager's bedtime forward by one hour. If the teenager then experiences an onset delay of 30 minutes or less, the behavioral module 304 may recommend moving the bedtime back by 30 minutes.

If the recommendation was not followed, the behavioral module 304 may instruct the user-interface module 308 to provide the user with some incentive to follow the recommendation. In some embodiments, the incentive is determined during a preference assessment and is modified based on the target subject's age. For example, a parent may provide information about how the target subject is motived by a type of candy, stickers, praise, etc. In another example, some children are motivated by charts, access to preferred activities (e.g., trip to the park with mom), attention, or a tangible reward. For example, the user-interface module 308 may provide the user with a $5 gift certificate as a tangible reward if the user subsequently follows the recommendation.

In some embodiments, if the behavioral module 304 determines that the recommendation was followed, the behavioral module 304 determines whether the target outcome was achieved. If the recommendation was followed but the target outcome was not achieved, the behavioral module 304 may generate a new recommendation. In some embodiments, if a user follows the recommendation multiple times and it does not help achieve the target outcome, the behavioral module 304 may investigate for procedural integrity and may suggest a new intervention or approach based on the feedback. The new intervention or approach is part of a larger plan for addressing a target outcome.

In some embodiments, the new recommendation may be selected based on a score associated with each of the recommendations. For example, if the behavioral module 304 scores different recommendations and only provides one of the recommendations, the behavioral module 304 may provide the second-best recommendation as a result of the first recommendation not working along with a modification to the overall plan for achieving the target outcome. For example, the first recommendation may be for an adult to not watch television within two hours of bedtime and the second recommendation may be for the adult to avoid reading books with alarming content, such as books about climate change.

In some embodiments, the behavioral module 304 may use the subsequent sensor data to add to the baseline for the behavioral patterns. For example, the behavioral module 304 revises the behavioral patterns with the subsequent sensor data after a user follows the recommendations and the target outcome is achieved.

In some embodiments, instead of a behavioral module 304 that applies rules for determining the behavior patterns of a set of sleep events of a target subject and determining a recommendation for achieving a target outcome, the sleeping application 103 includes a machine-learning module 306 that trains a machine-learning model to generate clusters of behavior patterns and output a recommendation for achieving the target outcome. In some embodiments, the machine-learning module 306 includes a set of instructions executable by the processor 335 to train a machine-learning model to output the recommendation for achieving the target outcome. In some embodiments, the machine-learning module 306 is stored in the memory 337 of the computing device 300 and can be accessible and executable by the processor 335.

In some embodiments, the machine-learning module 306 implements supervised learning by using a training dataset of initial sensor data with parameters, such as temperature, pressure, humidity, light, sound, motion, and thermal images as a function of time. The initial sensor data is labelled with the recommendation for achieving the target outcome. In some embodiments, the machine-learning module 306 implements unsupervised learning by using a training dataset of initial sensor data and does not include the labelled recommendation for achieving a target outcome. In some embodiments, the training datasets are further organized according to the attributes of the target subject, such as age, sex, weight, location, attachment style, target outcome, etc. Further organizing the data according to age may be advantageous because the sleep behavior of an infant, for example, is extremely different from the sleep behavior of an octogenarian. In some embodiments, the training data includes images of the physical environment and/or audio of the physical environment. In some embodiments, the machine-learning module 306 implements a deep learning machine-learning model.

In some embodiments, the machine-learning module 306 generates clusters for each parameter based on similarity of the data. For example, where the training data is labeled, the clusters may be for temperature, pressure, humidity, light, sound, motion, and thermal images as a function of time and may be further organized based on attributes, such as age, sex, weight, location, attachment style, target outcome, etc. In another example when the training data is unlabeled, the clusters may be for temperature, pressure, humidity, light, sound, motion, and thermal images as a function of time.

The output of the machine-learning model under training is a recommendation that can help achieve the target behavior. For example, in supervised learning, the model under training may be provided a time series of data from various sensors, with a first period during which a target behavior is not achieved and a second period during which the target behavior is achieved. The model may learn from the data various parameters that changed between the two periods. Each changed parameter may be a potential recommendation as part of the larger intervention. Based on such data, one or more parameters of the machine-learning model may be adjusted. The model may then be provided data only for the first period (during which target behavior is not achieved) and may generate a recommendation of a change to achieve the target behavior. The recommendation may be compared with a ground truth modification (e.g., reducing sound level; playing white noise; turning out the lights; etc.) that worked to achieve the target behavior. Feedback is provided to the machine-learning model, e.g., to adjust the weight on one or more nodes (if the model is implemented using a neural network). Training the model to make predictions for a large data set generates a trained machine-learning model that can then be used to make recommendations for real life subjects.

Once the machine-learning module 306 trains the machine-learning model, the trained machine-learning model receives initial sensor data for a target subject as well as attributes of the target subject. The machine-learning model outputs a recommendation for achieving the target outcome. In some embodiments, the machine-learning model may output a targeted behavior as well as a recommendation for how to achieve the targeted behavior.

In some embodiments, the machine-learning module 306 receives feedback that information about whether the recommendation was followed and, if so, whether implementing the recommendation led to achieving the target behavior. If the recommendation was followed and the recommendation did not help achieve the target behavior, in some embodiments, the machine-learning module 306 modifies the parameters of the machine-learning model accordingly.

The user-interface module 308 generates a user interface. In some embodiments, the user-interface module 308 includes a set of instructions executable by the processor 335 to generate the user interface. In some embodiments, the user-interface module 308 is stored in the memory 337 of the computing device 300 and can be accessible and executable by the processor 335.

The user-interface module 308 causes a user interface to be displayed that allows a user to enter information about the target subject. The information for the target subject may include a name, age, sex, weight, attachment style, etc. If the target subject is a baby, the user interface may include additional information related to a baby, such as the option to log feedings (breast feeding or formula), diaper changes, etc. In some embodiments, the user-interface module 308 generates a user interface to gather information about the bedtime routine that may be used to supplement the behavioral module's 304 analysis of behavior patterns. For example, a parent may describe that the bedtime routine includes nursing the baby in a rocker until the baby falls asleep. The behavioral module 304 may use this information to confirm sensor data identified by a motions sensor 250 that identifies the baby and the mother in the same location and moving in a repetitive motion as performing the nursing in a rocking chair part of the bedtime preparation.

In some embodiments, the user-interface module 308 generates a user interface for establishing user preferences including one or more target outcomes. For example, the user-interface module 308 may include questions about whether the user has a preferred temperature for the physical environment where the target subject is sleeping, a preferred humidity, a preferred brightness, a preferred sound level, etc. In some embodiments, the user interface may include suggestions, such as: “The American Medical Association recommends that sleep is best achieved in a room with a temperature of 68-72 degrees Fahrenheit. Is that okay for you?” In some embodiments, the user preferences include privacy settings. For example, a user may specify that they do not want images captured by the camera 260 due to medical concerns, military concerns, or other privacy issues. But the user may confirm that they are more comfortable with sensor data from the thermal imaging sensor 255.

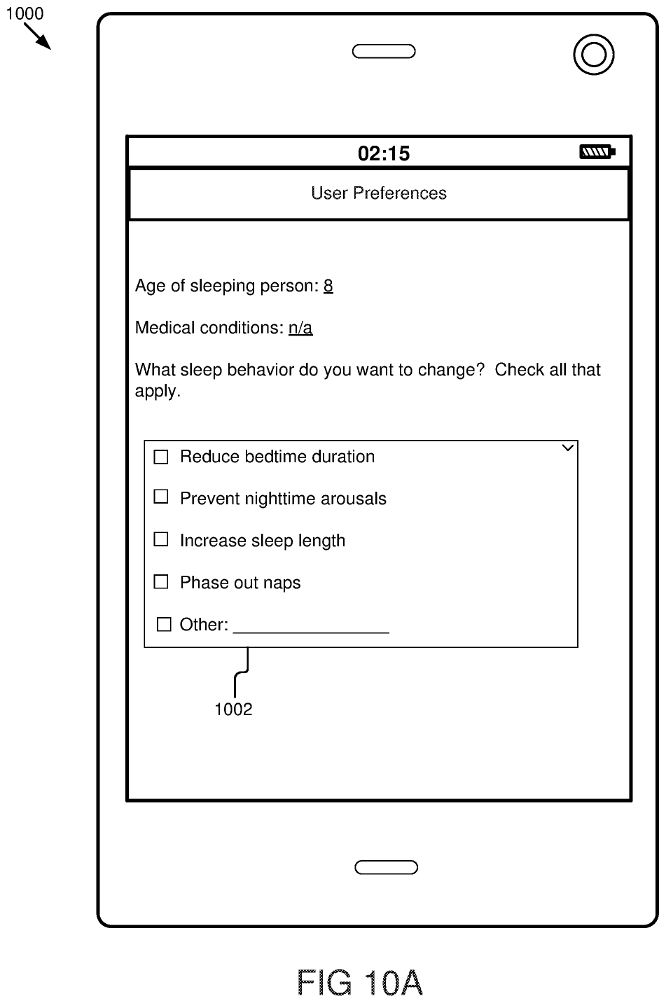

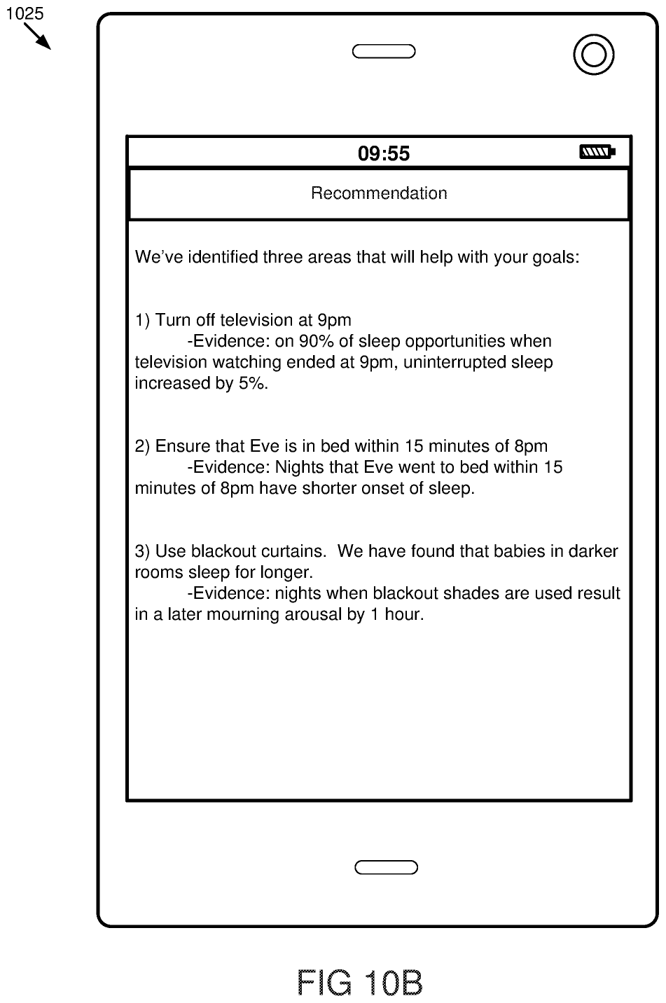

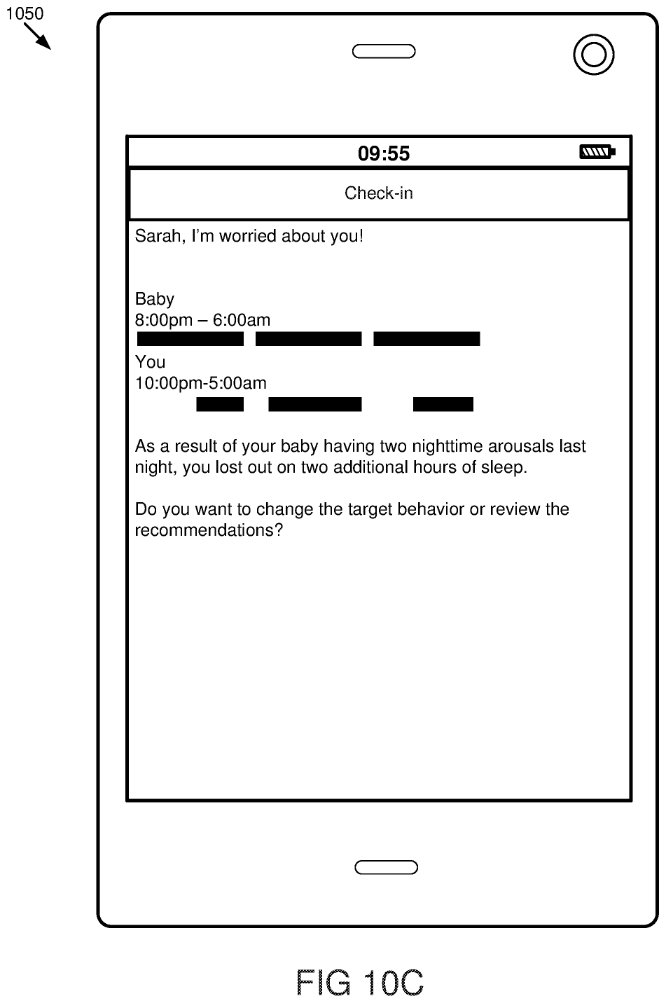

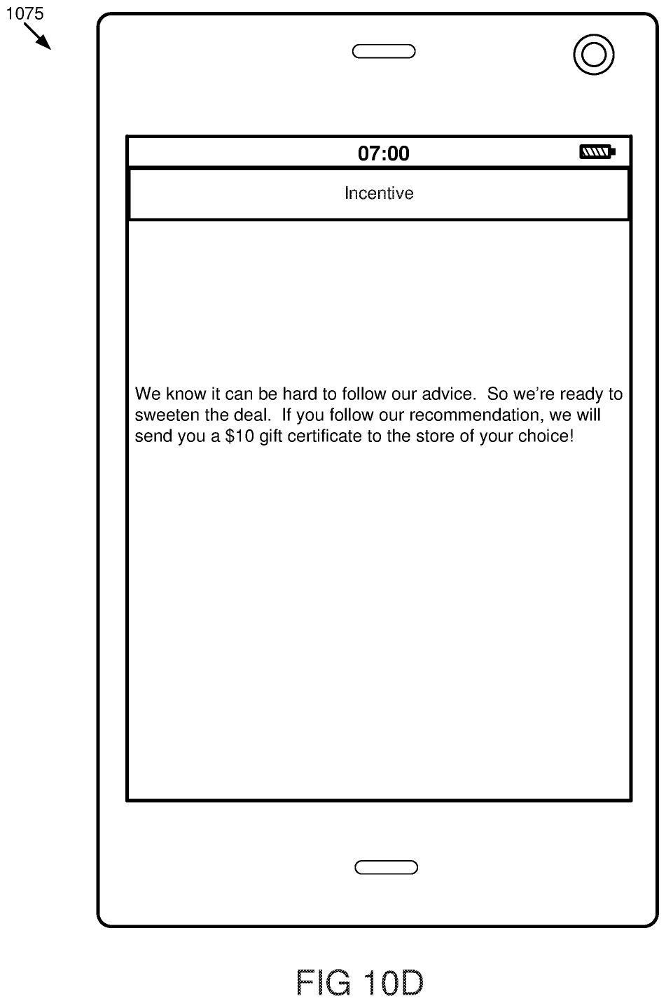

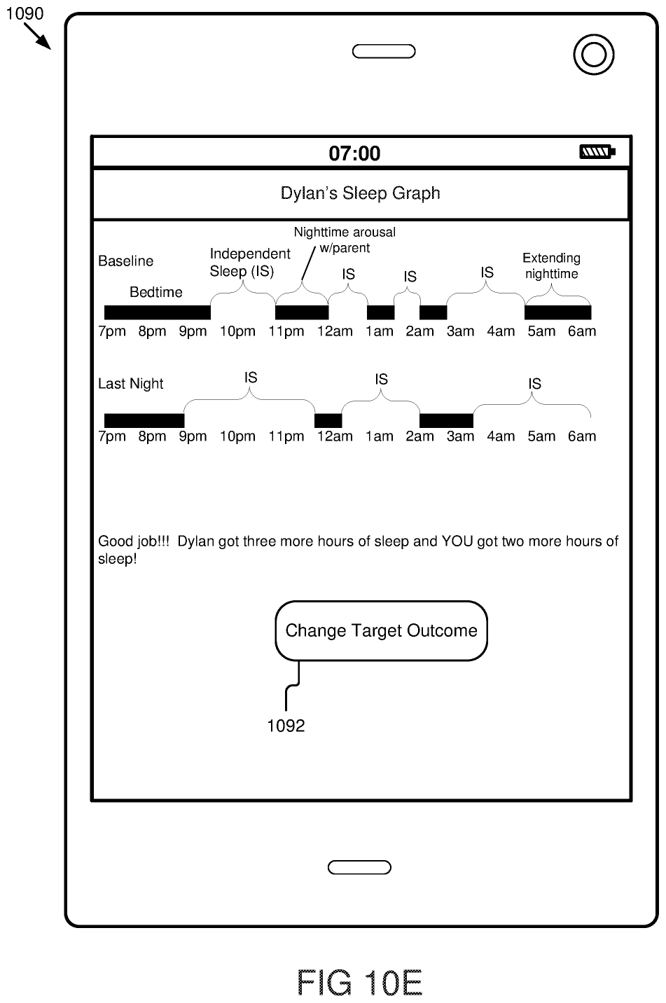

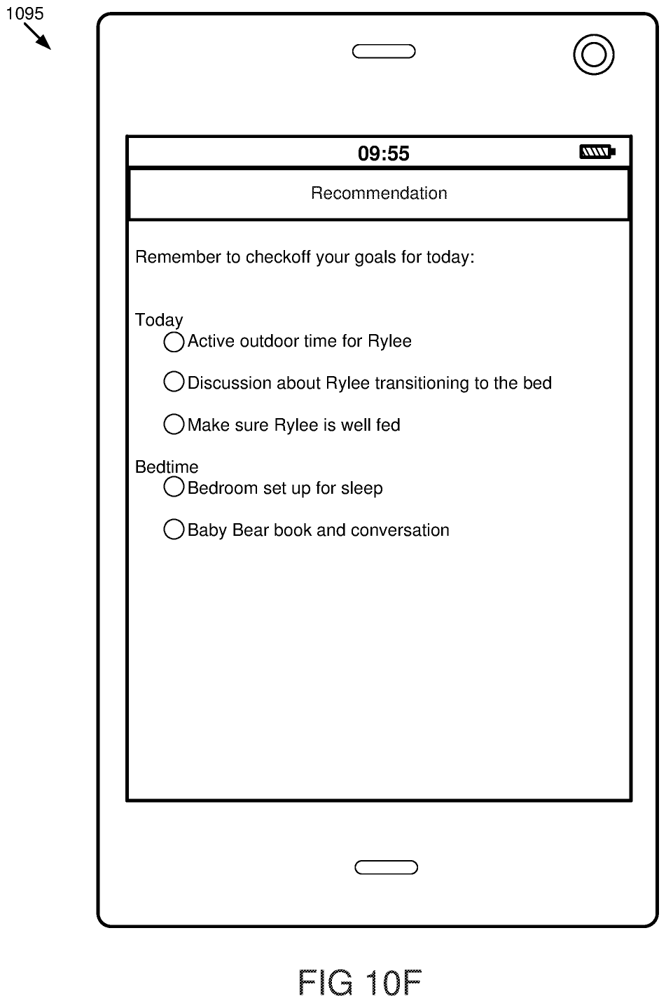

In some embodiments, the user-interface module 308 includes suggestions for target outcomes in a drop-down menu. Other options are possible, such as text fields where a user can enter data. Turning to FIG. 10A, an example user interface 1000 is illustrated for configuring sleep preferences. In this example, the user interface 1000 includes information already entered about the target subject, such as an age and medical conditions, and the target outcomes are listed as a drop-down menu 1002. In this example, a user selected target outcomes by adding checks to the boxes next to: reduce bedtime preparation, prevent nighttime arousals, increase sleep length, phase out naps, and an option to add an additional target outcome in a text field.

In some embodiments, the user-interface module 308 includes an option for suggesting targeted behavior to the user. For example, the machine-learning module 306 may receive the initial sensor data as input and output a targeted behavior, as well as a recommendation for achieving the targeted behavior, based on similarity of the initial sensor data to clusters of parameters.

In some embodiments, the user-interface module 308 includes an option for a user that is different from the target subject to add targeted behavior as well. For example, a parent may watch television after the baby falls asleep instead of going to sleep. The parent may benefit from a notification from the user-interface module 308 to go to bed earlier than usual.

FIG. 10B illustrates an example user interface 1025 for providing recommendations to achieve targeted behaviors. In this example, the recommendations are: turning off the television at 9 pm, ensuring that the child is in bed within 15 minutes of 8 pm, and that blackout curtains are used. Each recommendation is supported by data, based on the behavioral module 304 identifying behavioral patterns and determining the recommendations based on the behavioral patterns or the machine-learning model outputting the recommendations.

In some embodiments, the user-interface module 308 generates summaries of sleep changes over time for the target subject and a user if the user is different from the target subject.

FIG. 10C illustrates an example user interface 1050 for providing a health check-in to a user. In this example, the user-interface module 308 generates a comparison of the time that the target subject, i.e., the baby is sleeping as well as the parent. By illustrating the sleeping times together, the comparison acts as a visual aid to notify the user (grandparents, caregivers, spouses, etc.) of ways to help improve the primary nighttime caregiver's sleep. In this example, the comparison helps the user recognize that they need to get more sleep, times when more sleep is possible, and possibly follow the recommendations for achieving the target outcome or altering the target outcome to include fewer arousals.

FIG. 10D illustrates an example user interface 1075 for providing a tangible incentive for following the recommendation, according to some embodiments described herein. In this example, because the user has not followed the recommendation, the user-interface module 308 provides an offer of a $10 gift certificate to provide an additional incentive for the user to follow the recommendation.

A tangible reward may be helpful for an older target subject. For example, an athlete may benefit from a target outcome of getting enough sleep by following a recommendation for consuming enough protein and having a consistent bedtime.

In some embodiments, the user interface module 308 generates a visual analysis that includes a sleep summary for the user every morning to provide the user with a metric for gauging progress. FIG. 10E illustrates an example user interface 1090 for providing a sleep graph of a target subject, according to some embodiments described herein. In this example, the baseline sleep graph shows that the bedtime preparation lasts for two hours, sleep occurred from 9:30 to 11 pm, the baby experienced a nighttime arousal with parent from 11 pm-12 am, sleep occurred from 12 am-1 am, the baby experienced a nighttime arousal from 2 am-3 am, sleep occurred from 3 am-5 am, and although the baby was awake starting at 5 am, a parent was in the room with the baby trying to sleep with the baby to extend the nighttime while the parent slept.

The sleep illustrated in the graph is independent sleep, which is when the target subject sleeps with no help from any other people in the physical environment. In some embodiments, the processing module 302 determines times when the target subject was sleeping independently, when motions occurred, etc. The black sections are a mix of sleep arousal and adult interaction. The black sections illustrate when the baby is awake and are analyzed by the behavioral module 304 to determine what caused the sleep arousal. For example, for a baby, the sleep arousal may be caused by interaction with a parent but as the baby ages the sleep arousal may be caused by something else, such as a child accessing a tablet. The behavioral module 304 is able to identify the different causes of the sleep arousals based on the different types of sensor data. For example, the behavioral module 304 distinguishes between thermal sensor data that identifies the presence of different people in the room versus a child viewing a tablet, sounds produced by two people in the room versus a child tapping on a tablet, etc.

The user interface 1090 also shows the progress in improving sleep because during the last night, bedtime was reduced by 30 minutes, and the baby slept for three more hours. Although the summary does not include a sleep graph for the user, the user's sleep was also measured and the summary includes information about the parent's additional sleep as well. This is advantageous because when a baby causes a parent to rouse, the parent may have trouble getting back to sleep and may be doing things other than sleeping during the night because of the arousal, such as pacing around the living room, watching tv, and answering emails.

In this case, because the sleep is improving, the user interface includes a button 1092 for changing the target outcome. If the target outcome was reducing the bedtime preparation and increasing the sleep by three hours, the goal has been achieved and the user may be ready to establish a new goal. Examples of new goals may include falling asleep independently, achieving independent sleep throughout the entire night, rousing at a certain time in the morning, generalizing new behaviors to other caregivers and settings, and setting a goal over time to monitor for maintenance.

FIG. 10F illustrates an example user interface 1095 that includes a checklist of the recommendations, according to some embodiments described herein. The checklist may be particularly helpful for reminding a user about activities that are not as easily tracked. For example, this checklist includes ensuring active outdoor time for the target subject, having a discussion about the target subject transitioning to the bed, making sure that the target setup is well fed, setting up the bedroom for sleep, and reading and discussing a book designed to teach the target subject about sleeping alone.

In some embodiments, if the user provides input (e.g., a mouse with an arrow, a finger, etc.), the user interface includes an option to provide additional information about the subject. For example, clicking on “Active outdoor time for Rylee” may link to an article about the science behind sleep and its connection to activity. In another example, clicking on “Make sure Rylee is well fed” may link to a user interface that allows the user to input information about what Rylee ate and provide additional analysis about whether the amount of food is sufficient for a target subject of Rylee's age. In some embodiments, information about food consumption may be used by the behavioral module 304 to determine the target subject's eating patterns and may result in an additional recommendation being generated based on the user input.

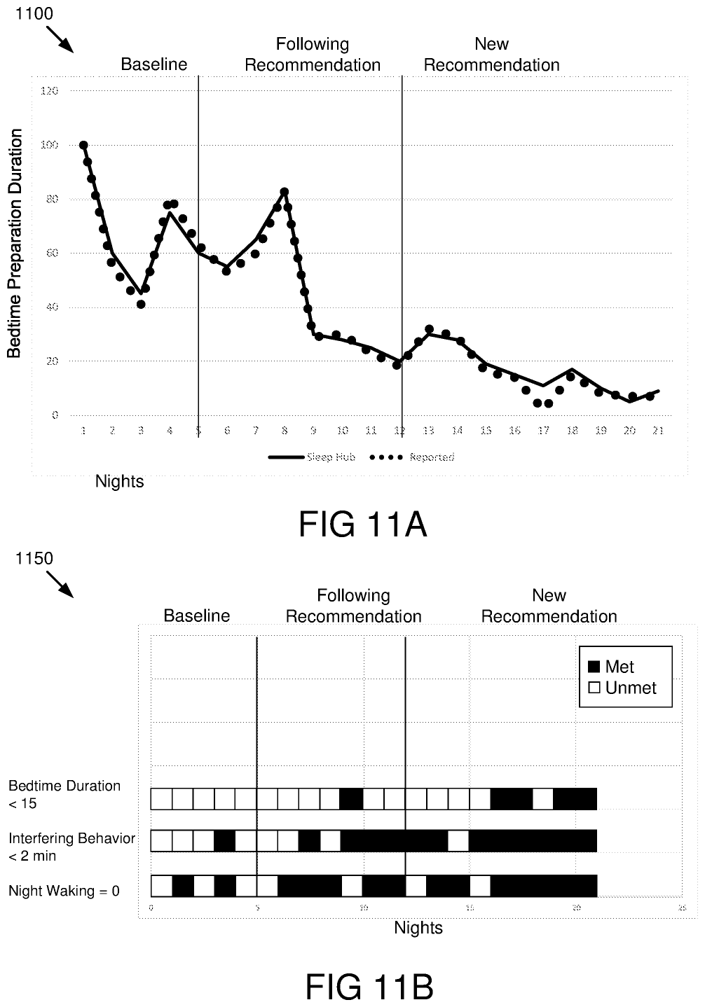

FIG. 11A illustrates an example graph 1100 of bedtime preparation as a function of nights based on a baseline stage, a stage when a recommendation is being followed, and a stage when a new recommendation is being followed, according to some embodiments described herein. In this example, the two lines represent data as determined by sensor data from the sleep hub 120 and reported by a user. The behavioral module 304 may determine that the time it takes to get the target subject ready for bed is longer than the self-reported information because the behavioral module 304 uses a different definition and/or self-reported information is more unreliable.

While the initial sensor data is being gathered to create a baseline during the first five days, a user is not implementing any recommendations. As a result, the bedtime preparation is quite long. The behavioral module 304 generates a recommendation that is followed by the user from nights five to 12; however, the recommendation is not successful is substantially improving the bedtime preparation length. As a result, the behavioral module 304 generates a new recommendation that is followed during nights 13 to 21. The new recommendation is much more effective in decreasing the bedtime preparation.

FIG. 11B illustrates an example graph 1150 of blocks where target outcomes are being achieved or not achieved during the stages illustrated in FIG. 11A, according to some embodiments. In this example, a solid block represents a night where a target outcome was achieved (“met”) and a white block represents a night where a target outcome was not achieved (“unmet”). The graph illustrates three target outcomes: a bedtime preparation less than 90 minutes (as illustrated in FIG. 11A), an interfering behavior less than two minutes, and no instances of night arousals. In this example, the behavioral module 304 generates multiple recommendations to address all of the target outcomes.

The user-interface module 308 generates a user interface that includes information about the personal information that is collected by the application, information about the user's right to delete personal information collected by the application, the right to opt-out of the sale of personal information, and the right of the user to be free from discrimination if the user chooses to invoke any of these rights. This information may be provided before the user enters any information.

The sleep assistant 310 provides sleep assistance to a user. In some embodiments, the sleep assistant 310 includes a set of instructions executable by the processor 335 to provide sleep assistance. In some embodiments, the sleep assistant 310 is stored in the memory 337 of the computing device 300 and can be accessible and executable by the processor 335.

In some embodiments, the sleep assistant 310 provides an interface for the sleep hub 120 when a user and/or target subject try to access functions on the sleep hub 120. For example, a sleep assistant 310 may enable the user to record audio of stories, songs, etc. to play during bedtime. In some embodiments, the sleep assistant 310 is connected to other services, such as a music-streaming application, a meditation application, etc. In another example, the user may request the sleep hub 120 to start the white noise machine via the sleep assistant 310. In some embodiments, the white noise machine continues to produce sound until the behavioral module 304 determines that sleep has ended. In some embodiments, the sleep assistant 310 includes an option to set the white noise machine on a timer or a manual option for turning it off.

In some embodiments, the sleep assistant 310 includes prompts for a user on the sleep event they are experiencing. The sleep assistant 310 may apply behavioral principles in an action-oriented mode designed to teach the user how to build healthy sleep habits built on science and support by medical experts. In some embodiments, the sleep assistant 310 asks the user for feedback on how they perceive the process, detects patterns, and gives advisory notifications to ensure a restful night. In some embodiments, the sleep assistant 310 includes a data repository with additional information about the science of sleep. For example, the sleep assistant 310 may instruct the user interface module 308 to generate a user interface for the user to search for specific articles, review a frequently asked questions section, etc.

In some embodiments, the sleep assistant 310 includes an option for triggering a conversation with a sleep consultant. The sleep consultant may be accessed through a live counseling video, through chat, etc. In some embodiments, the sleep assistant 310 also includes a chat bot option for answering common sleep questions by accessing a database with frequently-asked questions.

### Example Flowcharts

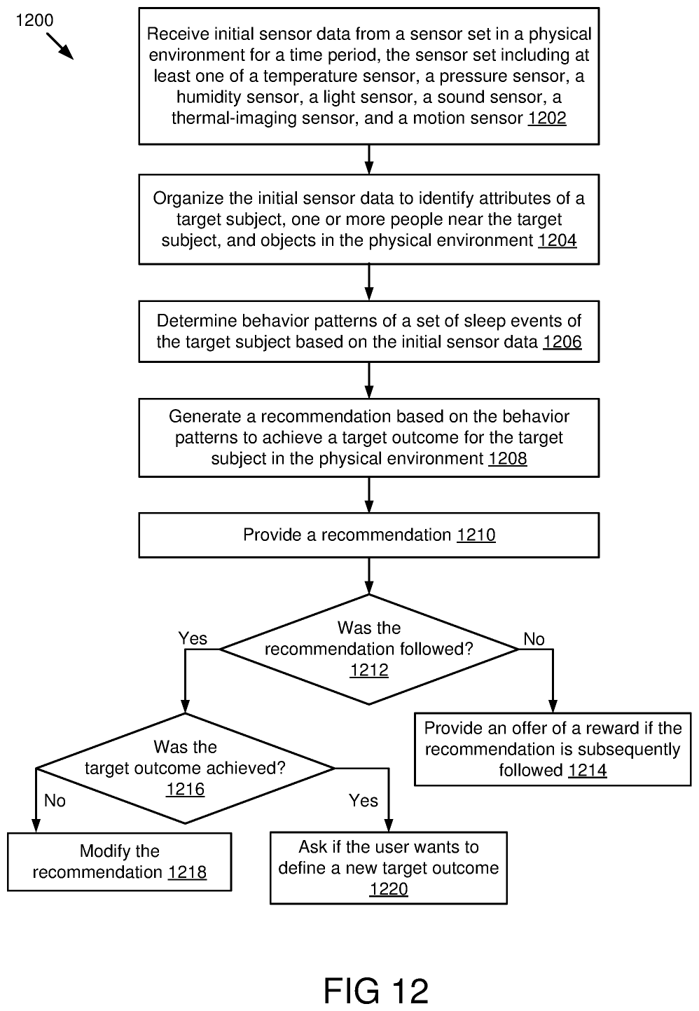

FIG. 12 is a flow diagram illustrating an example method for providing a recommendation to achieve a target outcome. The method illustrated in flowchart 1100 may be performed by the computing device 300 in FIG. 3.

The method 1200 may begin at block 1202. In block 1202, initial sensor data is received from a sensor set in a physical environment for a time period, the sensor set including at least one sensor selected from the set of a temperature sensor, a pressure sensor, a humidity sensor, a light sensor, a sound sensor, a thermal-imaging sensor, and a motion sensor. Block 1202 may be followed by block 1204.

At block 1204, the initial sensor data is organized to identify attributes of a target subject, one or more people near the target subject, and objects in the physical environment. Block 1204 may be followed by block 1206.

At block 1206, behavior patterns of a set of sleep events of the target subject are determined based on the initial sensor data. Block 1206 may be followed by block 1208.

At block 1208, a recommendation is generated based on the behavior patterns to achieve a target outcome for the target subject in the physical environment. Block 1208 may be followed by block 1210.

At block 1210, a recommendation is provided. Block 1110 may be followed by, block 1212.

At block 1212, it is determined whether the recommendation was followed. If the recommendation was not followed, block 1212 is followed by block 1214. At block 1214, an offer of a reward if the recommendation is subsequently followed is provided.

If the recommendation was followed, block 1212 is followed by block 1216. At block 1216, it is determined whether the target outcome was achieved. If the target outcome was not achieved, block 1216 is followed by block 1218. At block 1218, the recommendation is modified. For example, if the modification was to change the temperature in the room in order to reduce a nighttime arousal, the behavioral module 304 may change the recommendation for a different factor, such as reducing a number of times that the user enters the room after the baby is sleeping.

If the target outcome was achieved, block 1216 may be followed by block 1220. At block 1220, the user is asked if they want to define a new target outcome.

Various embodiments described herein include obtaining data from various sensors in a physical environment, analyzing such data, generating recommendations, and providing user interfaces. Data collection is performed only with specific user permission and in compliance with applicable regulations. The data are stored in compliance with applicable regulations, including anonymizing or otherwise modifying data to protect user privacy. Users are provided clear information about data collection, storage, and use, and are provided options to select the types of data that may be collected, stored, and utilized. Further, users control the devices where the data may be stored (e.g., client device only; client+server device; etc.) and where the data analysis is performed (e.g., client device only; client+server device; etc.). Data are utilized for the specific purposes as described herein. No data is shared with third parties without express user permission.

In the above description, for purposes of explanation, numerous specific details are set forth in order to provide a thorough understanding of the specification. It will be apparent, however, to one skilled in the art that the disclosure can be practiced without these specific details. In some instances, structures and devices are shown in block diagram form in order to avoid obscuring the description. For example, the embodiments can be described above primarily with reference to user interfaces and particular hardware. However, the embodiments can apply to any type of computing device that can receive data and commands, and any peripheral devices providing services.

Reference in the specification to “some embodiments” or “some instances” means that a particular feature, structure, or characteristic described in connection with the embodiments or instances can be included in at least one implementation of the description. The appearances of the phrase “in some embodiments” in various places in the specification are not necessarily all referring to the same embodiments.

Some portions of the detailed descriptions above are presented in terms of algorithms and symbolic representations of operations on data bits within a computer memory. These algorithmic descriptions and representations are the means used by those skilled in the data processing arts to most effectively convey the substance of their work to others skilled in the art, An algorithm is here, and generally, conceived to be a self-consistent sequence of steps leading to a desired result. The steps are those requiring physical manipulations of physical quantities. Usually, though not necessarily, these quantities take the form of electrical or magnetic data capable of being stored, transferred, combined, compared, and otherwise manipulated. It has proven convenient at times, principally for reasons of common usage, to refer to these data as hits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that, throughout the description, discussions utilizing terms including “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission, or display devices.

The embodiments of the specification can also relate to a processor for performing one or more steps of the methods described above. The processor may be a special-purpose processor selectively activated or reconfigured by a computer program stored in the computer. Such a computer program may be stored in a non-transitory computer-readable storage medium, including, but not limited to, any type of disk including optical disks, ROMs, CD-ROMs, magnetic disks, RAMs, EPROMs, EEPROMs, magnetic or optical cards, flash memories including USB keys with non-volatile memory, or any type of media suitable for storing electronic instructions, each coupled to a computer system bus.

The specification can take the form of some entirely hardware embodiments, some entirely software embodiments or some embodiments containing both hardware and software elements. In some embodiments, the specification is implemented in software, which includes, but is not limited to, firmware, resident software, microcode, etc.

Furthermore, the description can take the form of a computer program product accessible from a computer-usable or computer-readable medium providing program code for use by or in connection with a computer or any instruction execution system. For the purposes of this description, a computer-usable or computer-readable medium can be any apparatus that can contain, store, communicate, propagate, or transport the program for use by or in connection with the instruction execution system, apparatus, or device.

A data processing system suitable for storing or executing program code will include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

What is claimed is:

1. A computer-implemented method comprising:
receiving initial sensor data from one or more sensors of a sensor set in a physical environment for a time period, wherein the sensor set includes at least one of a temperature sensor, a pressure sensor, a humidity sensor, a light sensor, a sound sensor, a thermal-imaging sensor, and a motion sensor;
determining behavior patterns of a set of sleep events of a target subject based on the initial sensor data;
generating a recommendation based on the behavior patterns to achieve a target outcome for the target subject in the physical environment; and
providing the recommendation.

2. The method of claim 1, further comprising:
determining if the recommendation was followed; and
responsive to determining that the recommendation was followed, determining whether the target behavior was achieved.

3. The method of claim 2, further comprising:
responsive to determining that the target behavior was achieved, updating the behavior patterns based on subsequent sensor data; and
updating the target outcome based updating the behavior patterns.

4. The method of claim 1, further comprising:
determining if the recommendation was followed; and
responsive to the determining that the recommendation was not followed, providing an offer of a reward if the recommendation is subsequently followed.

5. The method of claim 1, wherein the sensor set includes the thermal-imaging sensor and the method further comprises:
detecting, based on the initial sensor data, the target subject, one or more people near the target subject, and one or more objects in the physical environment;
determining, based on the initial sensor data, a distance between the target subject and the one or more people;
determining a movement pattern of the target subject in the physical environment;
determining one or more movement patterns corresponding to the one or more people; and
determining one or more movement patterns for the one or more objects;
wherein determining the behavior patterns is based on the movement pattern of the target subject, the one or more movement patterns corresponding to the one or more people, and the one or more movement patterns for the one or more objects.

6. The method of claim 1, wherein the sensor set includes the sound sensor and the method further comprises:
detecting, based on the initial sensor data, a sound level in the physical environment;
detecting, based on the initial sensor data, sounds of the target subject, one or more people near the target subject, and other sounds in the physical environment;
filtering out specific sounds; and
determining sound patterns;
wherein determining the behavior patterns is based on the sound patterns.

7. The method of claim 1, wherein the sensor set includes the motion sensor and the method further comprises:
detecting, based on the initial sensor data, motion of the target subject, one or more people near the target subject, and one or more objects in the physical environment; and
determining a target subject movement pattern, a people movement pattern, and one or more object movement patterns;
wherein determining the behavior patterns is based on the target subject movement pattern, the people movement pattern, and the one or more object movement patterns.

8. The method of claim 1, wherein determining the behavior patterns includes determining attributes associated with at least one sleep event selected from the set of a bedtime preparation, a nighttime arousal, a naptime preparation, a naptime arousal, and sleeping.

9. The method of claim 1, further comprising:
providing a user interface that requests user preferences about the target outcome, wherein the target outcome is defined based on the user preferences.

10. The method of claim 1, further comprising:
receiving subsequent sensor data for a subsequent time period;
determining, based on a comparison of the subsequent sensor data to the behavior patterns, that an action is likely to precipitate a nighttime arousal or a naptime arousal; and
providing a warning that the action is likely to precipitate the nighttime arousal or the naptime arousal.

11. The method of claim 1, wherein the recommendation is provided to a user that is different from the target subject and the method further comprises:
determining behavior patterns of a set of sleep events of the user based on the initial sensor data.

12. The method of claim 11, further comprising:
receiving the initial sensor data associated with the user that identifies a length of time when the user is asleep; and
providing a user interface to the user that includes the length of time when the user is asleep as compared to when the target subject is asleep.

13. The method of claim 1, further comprising:
providing the initial sensor data as input to a trained machine-learning model; and
outputting, using the trained machine-learning model, the recommendation for achieving the target outcome.

14. A computing device comprising:
one or more processors; and
a memory coupled to the one or more processors, with instructions stored thereon that, when executed by the processor, cause the processor to perform operations comprising:
receiving initial sensor data from one or more sensors of a sensor set in a physical environment for a time period, wherein the sensor set includes at least one of a temperature sensor, a pressure sensor, a humidity sensor, a light sensor, a sound sensor, a thermal-imaging sensor, and a motion sensor; and
determining a baseline for one or more of a set of sleep events of a target subject based on the initial sensor data.

15. The computing device of claim 14, wherein the operations further comprise:
determining behavior patterns of a set of sleep events of a target subject based on the baseline for the one or more of the set of sleep events and the initial sensor data;
generating a recommendation based on the behavior patterns to achieve a target outcome for a target subject in the physical environment;
providing the recommendation;
determining if the recommendation was followed; and
responsive to determining that the recommendation was followed, determining whether the target behavior was achieved.

16. The computing device of claim 15, wherein the operations further comprise:
responsive to determining that the target behavior was achieved, updating the behavior patterns based on subsequent sensor data; and
updating the target outcome based updating the behavior patterns.

17. The computing device of claim 15, wherein the operations further comprise:
determining if the recommendation was followed; and
responsive to the determining that the recommendation was not followed, providing an offer of a reward if the recommendation is subsequently followed.

18. A non-transitory computer-readable medium with instructions stored thereon that, when executed by one or more computers, cause the one or more computers to perform operations, the operations comprising:
receiving initial sensor data from one or more sensors of a sensor set in a physical environment for a time period, wherein the sensor set includes at least one of a temperature sensor, a pressure sensor, a humidity sensor, a light sensor, a sound sensor, a thermal-imaging sensor, and a motion sensor;
determining behavior patterns of a set of sleep events of a target subject based on the initial sensor data;
generating a recommendation based on the behavior patterns to achieve a target outcome for a target subject in the physical environment; and
providing the recommendation.

19. The computer-readable medium of claim 18, wherein the operations further comprise:
determining if the recommendation was followed; and
responsive to determining that the recommendation was followed, determining whether the target behavior was achieved.

20. The computer-readable medium of claim 19, wherein the operations further comprise:
responsive to determining that the target behavior was achieved, updating the behavior patterns based on subsequent sensor data; and
updating the target outcome based updating the behavior patterns.
